(19) United States
(12) Patent Application Publication
(10) Pub. No.: US 2022/0160846 A1
(43) Pub. Date: May 26, 2022

(54) CARBAMOYL PHOSPHATE SYNTHATASE-1 FOR THE TREATMENT AND PREVENTION OF INTERNAL TISSUE INJURY

(71) Applicant: The Regents of the University of Michigan, Ann Arbor, MI (US)

(72) Inventors: Bishr Omary, Ann Arbor, MI (US); Min-Jung Park, Ann Arbor, MI (US)

(21) Appl. No.: 17/433,011

(22) PCT Filed: Feb. 27, 2020
(86) PCT No.: PCT/US2020/020083

§ 371 Date: Aug. 23, 2021

Related U.S. Application Data
(60) Provisional application No. 62/811,167, filed on Feb. 27, 2019.

(51) Int. Cl. A61K 38/53; A61P 29/00

(52) U.S. Cl. CPC A61K 38/53; A61P 29/00

(57) ABSTRACT

Provided herein are compositions methods for the treatment and/or prevention of tissue or internal organ injury. In particular, carbamoyl phosphate synthatase-1 (CPS-1) peptides and polypeptides (e.g., enzymatically active or inactive CPS-1 peptides and polypeptides), and methods of use thereof for the treatment and/or prevention of disease that results in tissue injury are provided.

### STATEMENT REGARDING FEDERAL FUNDING

This invention was made with government support under DK047918 awarded by the National Institutes of Health. The government has certain rights in the invention.

### FIELD

Provided herein are compositions methods for the treatment and/or prevention of internal tissue injury. In particular, carbamoyl phosphate synthatase-1 (CPS1) peptides and polypeptides (e.g., enzymatically active or inactive CPS1 peptides and polypeptides), and methods of use thereof for the treatment and/or prevention of internal tissue injury are provided.

### BACKGROUND

Many different proteins, called alarmins or damage-associated molecular patterns (DAMPs), are released during tissue injury and are involved in disease progression. For example, high-mobility group box-1 (HMGB1) is the prototypic DAMP protein released during diverse context of damage (Antoine D J et al. (2012) J Hepatol 56:1070-1079.; Ilmakunnas M. et al. (2008) Liver Transpl 14:1517-1525.; Kostova et al. (2010). Mol Cell Biochem 337:251-258.; Yan W et al. (2012) Hepatology 55:1863-1875.; herein incorporated by reference in their entireties). HMGB1 functions as a DNA chaperone in the nucleus; however, upon release, it triggers the secretion of pro-inflammatory cytokines through binding to toll-like receptor-4 (TLR4) or the receptor for advanced glycation end products (RAGE) (Bianchi Me. et al. (2017) Immunol Rev 280:74-82.; herein incorporated by reference in its entirety). Inner mitochondrial membrane cytochrome c is another protein found in the extracellular space under pathological condition with a potential role as a DAMP (Eleftheriadis et al. (2016) Front Immunol 7:279.; Miller T J et al. (2008) J Appl Toxicol 28:815-828.; herein incorporated by reference in their entireties). Moreover, DNA, RNA and mitochondrial DNA are also considered as DAMPs (Chen G Y, and Nunez G (2010) Nat Rev Immunol 10:826-837.; Szabo G and Petrasek J (2015) Nat Rev Gastroenterol Hepatol 12:387-400.; herein incorporated by reference in their entireties).

### SUMMARY

Provided herein are compositions methods for the treatment and/or prevention of internal tissue injury. In particular, carbamoyl phosphate synthatase-1 (CPS-1) peptides and polypeptides (e.g., enzymatically active or inactive CPS-1 peptides and polypeptides), and methods of use thereof for the treatment and/or prevention of internal tissue injury are provided.

In some embodiments, provided herein are compositions comprising a CPS1 polypeptide having at least 70% (e.g., 70%, 75%, 80%, 85%, 90%, 95%, 99%, 100%, or ranges therebetween) sequence identity (or similarity) to all or a portion of SEQ ID NO: 1, wherein the composition is not a product of nature, and wherein the CPS1 polypeptide exhibits a cytokine-like activity of wild-type of CPS1. In some embodiments, the CPS1 polypeptide comprises at least 70% (e.g., 70%, 75%, 80%, 85%, 90%, 95%, 99%, 100%, or ranges therebetween) sequence identity to one or a combination of SEQ ID NOs: 2-8 (e.g., at least 70% sequence identity with SEQ ID NOs: 2-7, 2-6, 2-5, 2-4, 2-3, 3-7, 3-6, 3-5, 3-4, 4-7, 4-6, 4-5, 5-7, 5-6, 2 and 4-7, 2 and 5-7, 2 and 6-7, 2 and 7, 2-3 and 5-7, 2-3 and 6-7, 2-3 and 7, 2-4 and 5-7, 2-4 and 6-7, 2-4 and 7, 2-5 and 7, 2-3 and 4-7, 2-3 and 5-7, 2-3 and 6-7, 2-3 and 7, and any other suitable combinations of included an excluded domains). In some embodiments, the CPS1 polypeptide lacks a portion comprising 25% or greater sequence identity to one or more of SEQ ID NOs: 2-8 (e.g., lacks SEQ ID NO: 2, 3, 4, 5, 6, or 7; lacks SEQ ID NOs: 2-3, 2-4, 2-5, 2-6, 3-4, 3-5, 3-6, 3-7, 4-5, 4-6, 4-7, 5-6, 5-7, 6-7, 2 and 5-7, 2 and 6-7, 2 and 7, 3 and 6-7, 3 and 7, 2 and 5, 3 and 5-7, 3 and 6-7, 3 and 5, 3 and 6, 4 and 7, 3 and 6, or any other combination of excluded domains).

In some embodiments, provided herein are compositions comprising a CPS1 peptide having at least 70% (e.g., 70%, 75%, 80%, 85%, 90%, 95%, 99%, 100%, or ranges therebetween) sequence identity (or similarity) with a portion of SEQ ID NO: 1 that is 8-30 amino acid residues in length, wherein the composition is not a product of nature, and wherein the CPS1 peptide exhibits a cytokine-like activity of wild-type of CPS1.

In some embodiments, the CPS1 peptide or polypeptide is fused to a second peptide or polypeptide. In some embodiments, the second peptide or polypeptide sequence is a carrier moiety, therapeutic moiety, or detectable moiety.

In some embodiments, (i) one or more of the amino acid residues in the CPS1 peptide or polypeptide are D-enantiomers, (ii) the CPS1 peptide or polypeptide comprises one or more unnatural amino acids, (iii) the CPS1 peptide or polypeptide comprises one or more amino acid analogs, and/or (iv) the CPS1 peptide or polypeptide comprises one or more peptoid amino acids. In some embodiments, the CPS1 peptide or polypeptide or an amino acid therein comprises a modification selected from the group consisting of phosphorylation, glycosylation, ubiquitination, S-nitrosylation, methylation, N-acetylation, C-terminal amidation, cyclization, substitution of natural L-amino acids with non-natural D-amino acids, lipidation, lipoylation, deimination, eliminylation, disulfide bridging, isoaspartate formation, racemization, glycation; carbamylation, carbonylation, isopeptide bond formation, sulfation, succinylation, S-sulfonylation, S-sulfinylation, S-sulfenylation, S-glutathionylation, pyroglutamate formation, propionylation, adenylylation, nucleotide addition, iodination, hydroxylation, malonylation, butyrylation, amidation, alkylation, acylation, biotinylation, carbamylation, oxidation, pegylation, and any other applicable peptide modification.

In some embodiments, the CPS1 peptide or polypeptide exhibits enhanced stability, solubility, cytokine-like activity, cell permeability, and/or bioavailability relative to SEQ ID NO: 1. In some embodiments, the CPS1 peptide or polypeptide lacks CPS1 enzymatic activity.

In some embodiments, provided herein are pharmaceutical compositions comprising a CPS1 peptide or polypeptide described herein and a pharmaceutically-acceptable carrier. In some embodiments, a pharmaceutical composition further comprises one or more additional therapeutic agents (e.g., for the treatment or prevention of acute internal tissue injury, for promoting tissue health, etc.).

In some embodiments, provided herein are methods of treating or preventing acute internal tissue diseases comprising administering to a subject a composition comprising a CPS1 peptide or polypeptide described herein. In some embodiments, provided herein are methods of treating or internal tissue injury comprising administering to a subject a composition comprising a CPS1 peptide or polypeptide described herein. In some embodiments, the subject suffers from an internal tissue injury or disease. In some embodiments, the subject has been subjected to a toxic or potentially toxic does of a drug or toxin (e.g., acetaminophen, alcohol, NSAIDS, statins, antibiotics, methotrexate or azathioprine, antifungals, niacin, steroids, allopurinol, antivirals, chemotherapeutics), herbal supplements (e.g., aloe vera, black cohosh, cascara, chaparral, comfrey, ephedra, kava, etc.), chemicals and solvents (e.g., vinyl chloride, carbon tetrachloride, paraquat, polychlorinated biphenyls, etc.), or radiation. In some embodiments, administering the composition increases tissue anti-inflammatory macrophage numbers and/or phagocytic activity. In some embodiments, administering the composition protects against internal tissue damage induced by apoptosis or other modes of cell death.

### BRIEF DESCRIPTION OF THE DRAWINGS

FIG. 1. CPS1 is released as a soluble multimeric protein that co-sediments with EVs. (A) Size measurement of primary mouse hepatocyte-derived EVs by NTA. The numbers show mean and mode sizes+/−standard error. (B) Immunoblotting of culture media or EVs from primary hepatocytes pre-incubated with saline or FL (0.5 μg/ml, 4 h). Coomassie brilliant blue staining (CBB) is shown as a loading control. (C) Immunoblotting of intact mouse serum or pelleted serum components was carried out from the indicated sedimentation fractions. Blotting was done using antibodies to the indicated proteins. Serum ALT is included at the bottom of the middle panels, in addition to a representative hematoxylin and eosin staining of the liver (arrows highlight areas of injury; bar=100 μm). (D) NTA of GFP-positive EVs isolated from primary hepatocytes that were transduced with lentiviral CPS1-GFP. The numbers show mean and mode sizes+/−standard error. (E,F) Sucrose gradient separation of the 100,000×g pellet from culture media of primary hepatocytes (E) or of rCPS1 (F). In panel F, 1% of starting material (rCPS1) was loaded in the last lane as a reference control.

FIG. 2. CPS1 is secreted through bile canaliculi. (A) Mouse hepatocytes were treated with saline or FL (0.5 μg/ml) for the indicated times. Abundance of CPS1, HMGB1 and LDH in the culture media, and of cleaved caspase 3 (c-Casp 3) in the cell lysates are shown. Coomassie brilliant blue stainings (CBB) are included to show equal protein loading. (B) Immunoblotting of mouse bile, serum and liver lysates using antibodies to the indicated antigens. The bile was collected at 20-minute intervals (#1-4 fractions). Similar results were observed from bile collections obtained from other mice. (C) Mouse bile was pelleted using the indicated g forces, followed by analysis of the pelleted and supernatant fractions by immunoblotting. (D) Bile was pelleted at 100,000×g, then the pellet and the supernatant fractions were analyzed by sucrose gradient sedimentation and immunoblotting similar to what was carried out in FIG. 1E,F. (E) Mouse liver was prepared for transmission electron microscopy imaging. Immunogold staining of the liver sections was carried out using rabbit anti-CPS1 antibody followed by goat anti-rabbit antibody conjugated with 10 nm gold particles. Arrowheads highlight CPS1 within the bile canaliculus (bar=200 nm); M, mitochondria. A negative control that did not include the anti-CPS1 antibody did not manifest a significant gold particle signal. (F) Mouse bile was obtained from the gallbladder (GB) or common bile duct (CBD) then immediately analyzed by SDS-PAGE followed by staining using Coomassie brilliant blue (CBB). Each lane of the gel was analyzed by mass spectrometry (see FIG. 12). (G) Abundance of urea cycle enzymes and other proteins were analyzed by immunoblotting using bile (pooled collection from CBD, lane 1) and total cell extracts from primary hepatocytes (two independent isolations, lanes 2 and 3). All the analyzed proteins were detected by mass spectrometry (as described in panel F and FIG. 12).

FIG. 3. CPS1 is taken up by mouse and human macrophages. (A) Serum from mice treated with FL was incubated at 37° C. for indicated time points, followed by immunoblotting using antibodies to the indicated proteins. (B) Primary endothelial cells were incubated with serum from a mouse pretreated with FL (0.15 mg/kg, 4 h). Immunoblot analysis of the cell lysates and the input serum was then carried out using antibodies to CPS1, albumin (as control serum protein) and vimentin (as control endothelial cell protein). (C) Jurkat cells were incubated with control media or conditioned media (CM) obtained from primary hepatocytes incubated with FL (0.5 μg/ml, 4 h). Immunoblot analysis of the Jurkat cell lysates and the input CM was then performed using antibodies to CPS1, albumin, and vimentin. CBB of a duplicate gel is included to profile the loaded proteins. (D) Peripheral blood mononuclear cells (PBMCs) from mice administered saline or FL were collected at 6 h or 10 h post-injection. Mϕ from the PBMC (PBMC-Mϕ) were analyzed by immunoblotting. Serum ALT levels are shown at the bottom. (E) J774 cells were cultured in their standard media (none), media for hepatocyte culture (media control) or CM from hepatocytes incubated with FL. Intracellular uptake of albumin (none detected) and CPS1 are shown. (F) Mice were administered recombinant (r)CPS1 followed by collection of 20 μl of blood at the indicated time point from tail (or heart at end of the experiment). The collected sera (2 μl) were then blotted with anti-CPS1 antibody and examined by CBB to ensure equal loading. (G) J774 cells were incubated with rTF (0.5 μg/ml) or rCPS1 (1 μg/ml). Immunofluorescence staining was done using antibodies to CPS1 and vimentin, followed by DAPI staining (nuclear). Arrows highlight CPS1 uptake in the cells. (H) Immunofluorescence staining of human PBMC-Mϕ pretreated with rTF or rCPS1, using antibodies to TF/vimentin and CPS1/vimentin followed by staining with DAPI. Arrows highlight rCPS1 uptake (bar=10 μm).

FIG. 4. CPS1 induces M2 polarization of macrophages. (A) Human Mϕ from PBMCs (hPBMC-Mϕ, n=5 per group) were treated with saline (control), rTF (0.5 μg/ml), rCPS1 (1 μg/ml), LPS (1 μg/ml) or IL-4 (20 ng/ml) for 24 h, followed by qPCR analysis of the indicated transcripts. *p<0.05,**p<0.01,***p<0.001. (B) Hepatic Mϕ (H-Mϕ) were isolated from mice injected with rTF (25 n=7) or rCPS1 (50 n=8) 24 h before the cell isolation, followed by qPCR analysis. (C) PBMCs were isolated from mice injected with rTF (25 μg) or rCPS1 (50 μg) then co-cultured in transwell plates (upper well) with naïve Kupffer cells isolated from other mice (lower well) (n=6 per group). After 24 h co-culture, Nos2 and Arg1 expression in the Kupffer cells was analyzed by qPCR. (D) Mice were injected with rTF (control, 24 h) or rCPS1 12 h or 24 h before sacrifice (n=3 per group). PBMC-Mϕ, bone marrow cells (BMC) and H-Mϕ were isolated from the mice and Arg1 mRNA was analyzed by qPCR. (E) PBMCs were isolated from mice injected with rTF or rCPS1, labeled with PKH26, then injected to another naive mouse. The H-Mϕ were isolated and stained with APC-labeled F4/80 antibody for flow cytometry analysis. The ratio of F4/80+ to PKH26− versus F4/80+ to PKH26+ cells (%) are included (red boxes). Right panel: immunofluorescence image of F4/80+ (green)/PKH26+ (red) cells in the liver co-stained with DAPI (blue) (bar=10 μm). Similar results were obtained in two other independent experiments. (F) J774 cells were treated with rTF (0.5 μg/ml), rCPS1 (1 μg/ml) or rCPS1 T471N (1 μg/ml, n=6 per group) for 24 h followed by qPCR analysis of Nos2 and Arg1 expression. N.S., not significant.

FIG. 5. FL-induced liver injury is attenuated by pre-injection of recombinant CPS1. (A) Mice were injected with rTF (25 μg) or rCPS1 (50 μg, 10 mice/group). After 24 h, each group was subdivided such 5 mice were injected with saline and 5 mice were injected with FL (0.15 mg/kg). Serum ALT was then measured (*p<0.05). (B) Representative hematoxylin and eosin staining of livers from the mice used in panel A (bar=200 (C,D) Immunoblot analysis of serum proteins (C) and liver lysates (D) from the 4 subgroups in panel A. The tested antigens are as indicated, together with CBB stainings as loading controls. c-Casp 3, cleaved caspase 3; c-Casp 7, cleaved caspase 7. (E-G) Livers from mice used in Panel A were subjected to TUNEL or immunofluorescence staining using antibodies to F4/80 or Ki-67. Positive cells were counted from five randomly-acquired images (HPF, high-power field). *p<0.05, ***p<0.001. (H) Hepatic Mϕ (H-Mϕ) were isolated from mice injected with rTF (n=3) or rCPS1 (n=3) followed by testing their phagocytosis capacity via uptake of FITC-labeled IgG-coated latex beads. F4/80-labeled (red, total H-Mϕ) or FITC-labeled (green, phagocytic H-Mϕ) were counted (bar=50 μm). HPF, high-power field. ***p<0.001. Note the marked increase in numbers of H-Mϕ and phagocytic H-Mϕ after rCPS1 administration to the mice.

FIG. 6. APAP-induced liver injury is attenuated by administration of rCPS1 prophylactically and therapeutically through Mϕs. (A-C) Male mice were injected via tail vein with rTF (25 μg) or rCPS1 (50 μg) 24 h prior to intraperitoneal APAP (350 mg/kg mouse weight) or saline administration. After 4 h, the mice [rTF-saline (n=4), rTF-APAP (n=7), rCPS1-saline (n=4), rCPS1-APAP (n=7)] were euthanized followed by analysis of the serum ALT (A, **p<0.01) and liver tissue histology using hematoxylin and eosin staining (B, bar=200 μm). Immunoblot analysis of the sera using antibodies to the indicated antigens is shown (C, CBB is included as a loading control). (D-F) Livers from panels A-C were subjected to TUNEL or immunofluorescence staining using antibodies to F4/80 or Ki-67. Quantification was carried out by counting positive-staining cells in a blinded fashion from five randomly-selected images from each liver (HPF, high-power field). *p<0.05, **p<0.01, ***p<0.001. (G-I) Mice were administered clodronate liposomes or PBS 48 h prior to injection of rTF or rCPS1. After another 24 h, the mice were given APAP or saline [control (n=4), clodronate (n=4), clodronate-APAP (n=5), clodronate-rTF-APAP (n=5), clodronate-rCPS1-APAP (n=5)]. F4/80 staining of liver and spleen showed Mϕ depletion in liver and spleen of clodronate-treated mice (G). Serum ALT levels and TUNEL staining of the livers are also shown (H,I). N.S., no significance between any two of groups 3-5. (J,K) Mice (n=30) were administered APAP intraperitoneally, followed 3 h later by tail vein injection with rTF or rCPS1 (15 mice/group). Serum was collected from the tail veins of the mice at 3 h intervals. Panel J shows the serum ALT levels, presented as percentage decrease compared with the values at the 3 h time point post APAP administration. *p<0.05. Panel H shows the immunoblot analysis of serum from 5 representative mice/group.

FIG. 7. Schematic model of CPS1 release and the biologic function of released CPS1. In normal liver, hepatic mitochondrial CPS1 is continuously secreted to bile (black arrows) while being undetectable in blood. Upon acute liver injury, CPS1 is released to the bloodstream where it is rapidly taken up by circulating monocytes and leads to their M2 polarization and homing to the liver independent of CPS1 enzyme activity (blue arrows). The endogenous CPS1-induced anti-inflammatory properties of M2-Mϕ protection from liver injury can also be provided therapeutically upon administration of recombinant CPS1 in experimental APAP-induced liver injury.

FIG. 8. CPS1 release in hepatocytes ex vivo is distinct from the other secretome components. (A) Primary cultured mouse hepatocytes were pre-incubated with brefeldin A (BFA, 2 μg/ml) or Exo1 (2 followed by treatment with fas ligand (FL) (4 h, 0.5 μg/ml). The media or the cell fraction were immunoblotted with antibodies to the indicated antigens. Cyt c, cytochrome c; c-Casp 3, cleaved-caspase 3. (B) Hepatocytes were incubated with FL, rotenone (10 μM) or glucose oxidase (10 mU/ml) for 4 h. Hepatocyte lysates or the culture media were then immunoblotted with antibodies to the indicated antigens. Ponceau S stain of the blot is included to show the protein loading. For immunoblots of the media, the antigens were selected to represent different subcellular compartments (listed to the right of the panel). (C) Primary mouse hepatocytes were incubated with saline or FL followed by isolation of the culture media then pelleting using the indicated speeds. The pellet or supernatant fractions were blotted with antibodies to the indicated antigens. The culture media before pelleting (‘Starting’) is included as control. (D,E) Mouse hepatocytes were pre-incubated with GW4869 (GW, 5 or 50 amiloride (Ami, 15 or 150 nM), fausdil (Faus, 1, 10 or 100 μM) or Y-27632 (Y27, 1 or 10 μM) for 1 h, followed by FL treatment (0.5 μg/ml) for 4 h. The culture media and the hepatocyte lysates were then immunoblotted using antibodies to the indicated antigens. Coomassie brilliant blue stainings (CBB) are included to show equal protein loading.

FIG. 9. CPS1 is released as a soluble protein that aggregates into multimers. Mice were administered FL to induce liver injury, followed by collection of serum. (A) Sucrose gradient centrifugation of the 100,000×g pellet or supernatant fractions of mouse serum. A total of 12 fractions were collected from sucrose gradient column, followed by immunoblotting of the fractions using anti-CPS1 antibody. (B) Transmission electron microscopy of the 100,000×g pellet obtained from mouse serum. Negative staining shows typical exosomes in the pellet fraction. Immunogold staining using 10 nm gold particles conjugated to anti-CPS1 antibody showed gold particles that colocalized with irregular structures presumed to be CPS1 aggregates/multimers. Scale bars=100 nm.

FIG. 10. Generation of recombinant wild type and mutant human CPS1 proteins in insect cells, and their enzymatic activities. (A, B and C) CBB staining of recombinant (r) His-tagged proteins purified using Ni-nitrilotriacetic acid (NTA) agarose, and immunoblots of duplicate SDS-PAGE gels using antibodies to CPS1 and transferrin (TF). (A) rHis-CPS1, N-terminal His-tagged recombinant human wild-type CPS1; (B) rHis-TF, N-terminal His-tagged recombinant human transferrin mutant; (C) rHis-CPS1 T471N, the His-tagged recombinant T471N mutant which is known to inactivate CPS1 activity in patients. (D) Enzymatic activity of rCPS1 (15 μg) was measured by determining carbamoyl phosphate produced by converting it to hydroxyurea.

FIG. 11. CPS1 is secreted through bile canaliculi in humans. Immunoblot analysis of human bile collected from four independent patients (#1-4, left panel). The right panel shows the bile samples from patient #4 that were collected at 1-minute consecutive intervals. Bile samples were subjected to acetone precipitation followed by reconstitution of the pellets with sample buffer. Equal bile fractions were loaded per lane then analyzed by immunoblotting using antibodies to CPS1 or transferrin, or by Coomassie brilliant blue staining (CBB).

FIG. 12. Categorization and validation of bile proteins identified by mass spectrometry. (A) Proteins identified by mass spectrometry were categorized by the PANTHER (protein annotation through evolutionary relationship) classification system (www.pantherdb.org). Among the cellular organelle components, 20.7% of the proteins were mitochondrial. (B) Bile samples obtained directly from mouse gallbladder (GB) or by cannulation of the common bile duct (CBD) were analyzed by immunoblotting using antibodies to the indicated antigens. A sampling of the proteins analyzed included those that were identified by mass spectrometry as present in the GB and CBD, in the GB alone, or in the CBD alone. CPS1, transferrin and albumin were detected by mass spectrometry at 125, 10, or 160 times relative fold, respectively, in CBD versus GB bile; and chymotrypsin was at 45 times relative fold in GB vs CBD bile. EF-Tu, elongation factor thermo unstable; AASS, aminoadipate-semialdehyde synthase; PCCA, propionyl-CoA carboxylase alpha subunit.

FIG. 13. Bile CPS1 is rapidly degraded. (A) Mouse bile was incubated at 37° C. for the indicated time points followed immediately by adding SDS-containing sample buffer, separation by SDS-PAGE then immunoblotting using antibodies to CPS1, transferrin and albumin. CBB of the analyzed bile samples is included to show protein loading. (B) Quantification of the relative band intensities of CPS1 from three independent experiments using three separate bile specimens isolated from mouse common bile duct. (C) Abundance of chymotrypsin and elastase in the first and sixth fraction of mouse bile collected at 20-minute intervals. The first fraction contains relatively high levels of chymotrypsin which leads to degradation of CPS1 and explains its limited detection in bile stored in the GB (as compared with freshly isolated bile obtained from the CBD after washout of bile that may already be present in the biliary system).

FIG. 14. CPS1 indirectly induces M2 polarization of hepatic macrophages. (A) Hepatic Mϕ (H-Mϕ) were isolated and incubated with rTF (0.5 μg/ml), rCPS1 (1 μg/ml), LPS (1 μg/ml) or IL-4 (20 ng/ml) for 24 h, followed by qPCR analysis (*p<0.05, ***p<0.001). (B) H-Mϕ were isolated from mice injected with rTF or rCPS1 24 h before, followed by immunoblotting of the lysates with antibodies to p-Stat6 (Tyr 641) or total Stat6. The CBB staining of a parallel gel is included as a loading control.

FIG. 15. CPS1 decreases the expression of Cxcr2 and Ccr1 in hepatic macrophages. (A) Mice were administered rTF or rCPS1 (n=4 per group), followed by analysis of the hepatic Mϕ gene expression using microarrays. The iPathwayGuide software analysis indicated that the genes related with chemokine signaling were altered by rCPS1 administration compared to rTF, and expression of Cxcr2 and Ccr1 were most highly decreased in hepatic Mϕ of the rCPS1-injected mice. (B) Hepatic Mϕ were isolated from normal mice and incubated with IL-4 for 24 h, followed by qPCR analysis of Cxcr2 and Ccr1 mRNA. (C) mRNA levels of Cxcr2 and Ccr1 genes were analyzed in the hepatic Mϕ isolated from mice injected with rTF or rCPS1 (*p<0.05, **p<0.01).

FIG. 16. Experimental flow chart for testing homing of PBMC-Mϕ to the liver. PBMCs were isolated from mice 12 h after administering rTF (25 μg/kg) or rCPS1 (50 μg/kg). The cells were stained with the dye PKH26 (that labels cell membranes), followed by tail-vein injection into new mice. After 24 h, H-Mϕ were isolated from the liver, labeled with APC-conjugated F4/80 antibodies, then subjected to flow cytometry to measure the ratio of PKH26⁺ and/or F4/80⁺ cells in each mouse liver.

FIG. 17. Circulating CPS1 primes endogenous hepatic macrophages to release cytokines that protect hepatocytes ex vivo from FL-induced cell death. (A) Experimental flow chart. Female mice were injected with rTF (25 μg/kg) or rCPS1 (50 μg/kg) via tail vein, followed by administration of APAP (450 mg/kg) intraperitoneally to boost the hepatic Mϕ (H-Mϕ) before their isolation. Conditioned media (CM) was obtained from the H-Mϕ cultured for 24 h, which was then tested for its protective effect on primary hepatocytes treated with FL (0.15 mg/kg). (B) Percentages of hepatocyte cell death as determined by staining with 0.04% trypan blue. Data are presented as mean±standard deviation (**p<0.01). (C) Immunoblot analysis of hepatocyte lysates obtained from the experiment described in panel A. c-Casp 3, cleaved caspase 3; c-Casp 7, cleaved caspase 7; c-K18, cleaved keratin 18 (a down-stream caspase substrate).

FIG. 18. Comparison of ALT levels of mice in the presence or absence of rTF. More than 10 mice per group were treated with rTr or saline intravenously followed administration of APAP or FL as described in Materials and Methods. Sera were analyzed for ALT. Note that rTF injection did not promote or protect from liver injury in all paired comparisons saline, FL, and APAP groups (+/−rTF).

FIG. 19. Exemplary truncated CPS1 polypeptides and polypeptide variants.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a CPS1 peptide” is a reference to one or more CPS1 peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of and/or” consisting essentially of embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., CPS1 peptide or polypeptide and a second agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “treating” refers to inhibiting a disease, disorder or condition (e.g., acute internal tissue injury) in a subject. Treating the disease or condition includes ameliorating at least one symptom, reducing severity, impeding progress, and/or curing the subject of the disease or condition.

As used herein, the term “preventing” refers to prophylactic steps taken to reduce the likelihood of a subject (e.g., an at-risk subject, a subject suffering from acute internal tissue injury) from contracting or suffering from a particular disease, disorder or condition. The likelihood of the disease, disorder or condition occurring in the subject need not be reduced to zero for the preventing to occur; rather, if the steps reduce the risk of a disease, disorder or condition across a population, then the steps prevent the disease, disorder or condition within the scope and meaning herein.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are typically of about 30 amino acids or less in length (e.g., 30, 25, 20, 15, 10, 6, or less, or ranges therebetween (e.g., 6-30)). A peptide may comprise natural amino acids, non-natural amino acids, amino acid analogs, and/or modified amino acids. A peptide may be a subsequence of naturally occurring protein or a non-natural (artificial) sequence.

As used herein, the term “peptoid” refers to a class of peptidomimetics where the side chains are functionalized on the nitrogen atom of the peptide backbone rather than to the a-carbon.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence “having at least Y % sequence identity with SEQ ID NO:Z” may have up to X substitutions relative to SEQ ID NO:Z, and may therefore also be expressed as “having X or fewer substitutions relative to SEQ ID NO:Z.”

As used herein, the term “wild-type,” refers to a gene or gene product (e.g., protein) that has the characteristics (e.g., sequence) of that gene or gene product isolated from a naturally occurring source, and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature, but have altered sequences when compared to the wild-type gene or gene product; they are not the most commonly occurring sequence. “Synthetic” mutants are genes or gene products that have altered sequences when compared to the wild-type gene or gene product and do not occur in nature. Mutant genes or gene products may be naturally occurring sequences that are present in nature, but not the most common variant of the gene or gene product, or “synthetic,” produced by human or experimental intervention.

As used herein, the term “full-length CPS1 protein” refers to the wild-type CPS1 sequence (SEQ ID NO: 1), naturally-occurring mutant versions thereof, and synthetic or modified (i.e., non-naturally occurring) versions thereof, that maintain a specified structural and/or functional characteristic of the wild-type CPS1 (e.g., anti-inflammatory protective cytokine functionality).

As used herein, the terms “CPS1 polypeptide” and “CPS1 peptide” refer to fragments of the full-length wild-type CPS1 sequence (SEQ ID NO: 1), naturally-occurring mutant versions thereof, and synthetic or modified (i.e., non-naturally occurring) versions thereof, that maintain a specified structural and/or functional characteristic of the wild-type CPS1 (e.g., anti-inflammatory protective cytokine functionality).

As used herein, “acute tissue injury” is the rapid (e.g., <14 days) appearance of severe symptoms and complications of a disease or condition.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

### DETAILED DESCRIPTION

Provided herein are compositions methods for the treatment and/or prevention of internal tissue injury. In particular, carbamoyl phosphate synthatase-1 (CPS-1) peptides and polypeptides (e.g., enzymatically active or inactive CPS-1 peptides and polypeptides), and methods of use thereof for the treatment and/or prevention of internal tissue injury are provided.

Experiments were conducted during development of embodiments herein demonstrate uptake of serum CPS1 by macrophages; M2 polarization of monocytes and hepatic macrophages by CPS1, independent of its enzymatic activity, and prophylactic/therapeutic effects of rCPS1 in an experimental tissue injury model. Experiments were conducted using liver tissue as an experimental system; however, embodiments herein are not limited to liver tissue. Embodiments herein find use in treatment/prevention of tissue injury (e.g., internal tissue injury, acute tissue injury, etc.) in a variety of tissues and diseases/conditions described herein.

CPS1 is a readily detected protein released from apoptotic hepatocytes (Weerasinghe S V, et al. (2014) Am J Physiol Gastrointest Liver Physiol 307:G355-364.; herein incorporated by reference in its entirety). Experiments conducted during development of embodiments herein demonstrate that CPS1 is non-classically secreted, and its release occurs regardless of liver damage but through different routes depending on the presence or absence of liver damage. Biliary release of CPS1 was an unexpected finding along with the identification of several other mitochondrial proteins in bile. The rapid clearance of serum CPS1 occurs via uptake by circulating monocytes, which in turn elicits their activation into anti-inflammatory cells that home to the liver and protect from liver injury induced by FL or APAP. The cytokine-like anti-inflammatory promoting effect of CPS1 is enzyme-independent and involves PBMC-Mϕ and bone marrow cells, though immune cells in other tissue compartments may be potential targets.

Most cells, including hepatocytes, release context-dependent EVs. CPS1 was previously reported as a component of cultured rat hepatocyte-derived exosomes based on proteomic findings (Conde-Vancells J et al. (2008) J Proteome Res 7:5157-5166.; herein incorporated by reference in its entirety). However, experiments conducted during development of embodiments herein indicate that CPS1 that is collected using standard exosome preparation methods primarily partitions with the exosome fraction because of its propensity to oligomerize and form multimers rather than being a component of EVs. This is based on biochemical assessment after sucrose gradient sedimentation of rCPS1 or CPS1 found in blood or bile, or upon ultrastructural evaluation. It is contemplated that the aggregation propensity of CPS1 increases its accessibility, recognition and uptake by pino-/phago-cytosis of Mϕ or through a receptor mediated uptake.

Experiments conducted during development of embodiments herein identified biliary secretion of CPS1. While non-protein bile components are well characterized, only a limited number of bile proteins have been identified due to technical challenges (Farina A, et al. (2009) Expert Rev Proteomics 6:285-301.). Proteomic analysis has identified several proteins in human bile (Barbhuiya M A et al. (2011) Proteomics 11:4443-4453.; Guerrier L et al. (2007) J Chromatogr A 1176:192-205.; Zhang D et al. (2013) PLoS One 8:e54489.; Zhou H et al. (2005). Rapid Commun Mass Spectrom 19:3569-3578.; Farina A et al. (2009) J Proteome Res 8:159-169.; Kristiansen T Z et al. (2004). Mol Cell Proteomics 3:715-728.; herein incorporated by reference in their entireties). Experiments conducted during development of embodiments herein demonstrate that CPS1 is found in both mouse and human bile; the detection of CPS1 by immunoblotting of human bile was feasible using fresh bile and acetone precipitation of the protein fraction, while detection of CPS1 in mice bile did not require acetone precipitation. Even short-term storage of human bile at −80° C. leads to degradation and loss of CPS1 detection.

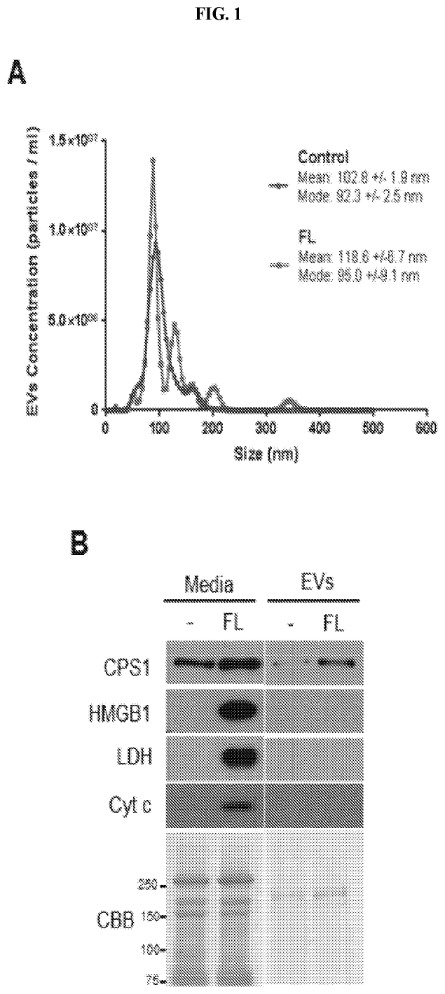

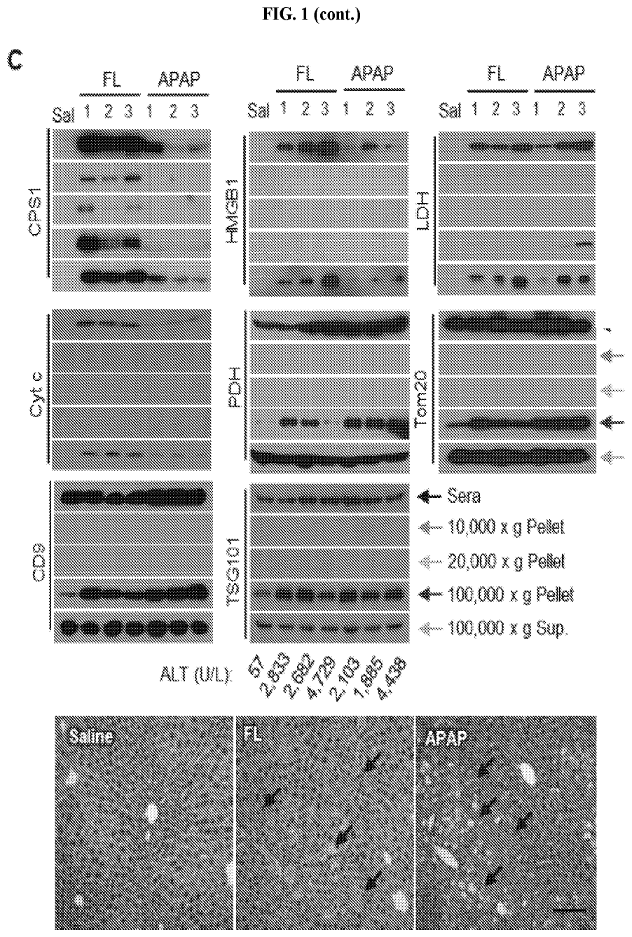

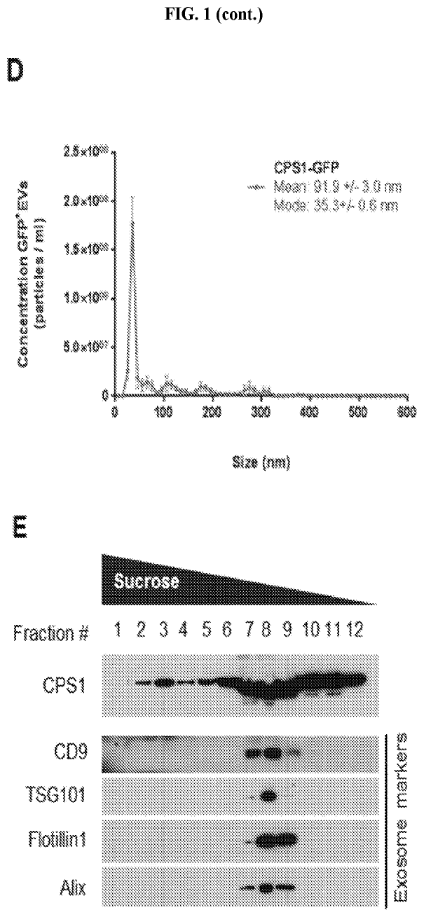

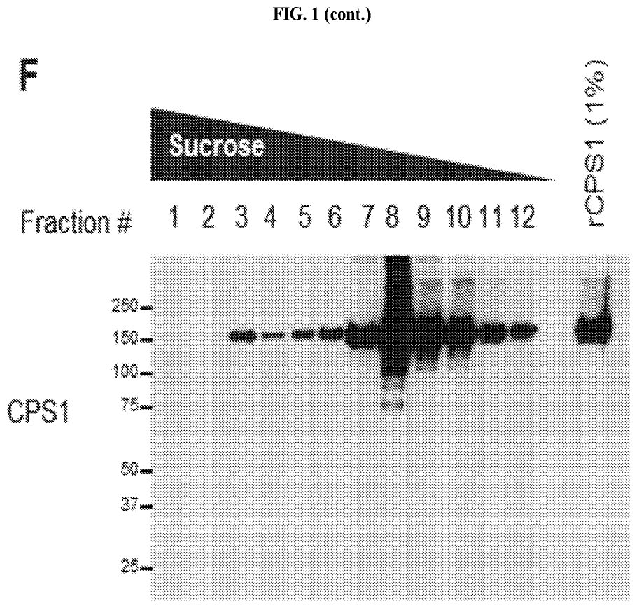

The stoichiometry of CPS1 that is released into bile compared to total liver CPS1 is very small. It is estimated that 0.002% of mouse liver CPS1 is excreted into bile per hour (based on sequential collection of bile followed by immunoblotting). Although the relative bile content of CPS1 is very small compared to hepatocytes, the absolute amount is not be trivial given the abundance of CPS1. The amount of CPS1 released to culture media of healthy primary mouse hepatocytes is comparable to the amount released by dying cells when other damage marker proteins also become readily detectable (FIGS. 1B, 2A, and 8).

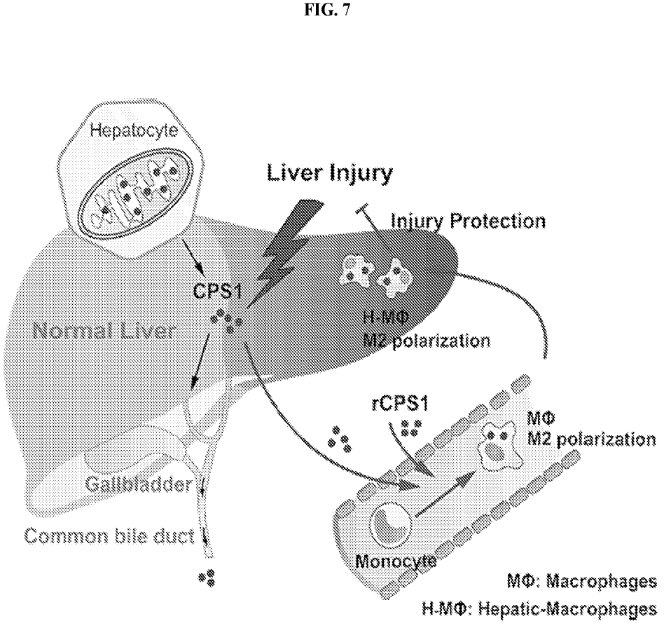

The liver serves an immune function through hepatocyte secretion of specific proteins into blood (Zhou Z, et al (2016) Cell Mol Immunol 13:301-315.; herein incorporated by reference in its entirety), and harbors the largest number of Mϕ (˜80% of body's total) (Krenkel O and Tacke F (2017) Nat Rev Immunol 17:306-321.; herein incorporated by reference in its entirety). Proteins released from hepatocytes into sinusoids could interact and communicate with liver-resident Kupffer cells and circulating monocytes. Kupffer cell activation and recruitment of circulating monocytes have been demonstrated in mice administered with APAP (Antoniades C G et al. (2012) Hepatology 56:735-746.; Holt M P, et al. (2008) J Leukoc Biol 84:1410-1421.; Zigmond E et al. (2014) J Immunol 193:344-353.; Ju C et al. (2002) Chem Res Toxicol. 15:1504-1513.; You Q et al. (2013) Biochem Pharmacol. 86:836-843.; herein incorporated by reference in their entireties). In addition, the presence of human Mϕ within an anti-inflammatory/regenerative microenvironment of the liver was observed in patients with APAP-induced acute liver failure, suggesting a beneficial effect of hepatic Mϕ (Antoniades C G et al. (2012) Hepatology 56:735-746.: herein incorporated by reference in its entirety). In line with these observations, circulating CPS1 protected from APAP- or FL-induced liver damage, at least partially, via triggering M2 polarization of bone marrow-derived monocytes and hepatic Mϕ in a CPS1 enzyme-independent manner. In addition to IL-4 and IL-13 that induce M2 polarization through IL-4R, findings during development of embodiments herein highlight CPS1 as another potential M2 inducer. Considering that 10-50 ng/ml of IL-4 is generally used to induce M2 polarization ex vivo, the stoichiometry of rCPS1 (1 μg/ml) is comparable to IL-4 (after correcting for the much smaller IL-4 protein size), albeit the precise mechanism underlying Mϕ activation by CPS1 remains to be elucidated. Like HMGB1, which triggers the release of pro-inflammatory cytokines via TLR4 or RAGE binding (Bianchi Me. et al. (2017) Immunol Rev 280:74-82.; herein incorporated by reference in its entirety), CPS1 may be recognized by a specific receptor on Mϕ and may signal through phagocytosis dependent or independent modes. Taken together, the findings (FIG. 7) show that the mitochondrial protein CPS1 is normally released into bile and demonstrate a direct anti-inflammatory M2 polarization effect of CPS1 that is independent of its enzyme activity. The ability of rCPS1 that is administered intravenously after injury to ameliorate APAP-induced liver injury raises the exciting possibility of its utility as a therapeutic in select cases of acute tissue injury or disease.

Provided herein are compositions (e.g., CPS1-derived proteins, polypeptides, peptides, fusions, etc.) that protect against liver damage (e.g., apoptotic and nonapoptotic liver damage, drug-induced liver damage, pathogen-induced liver damage, metabolic and other liver diseases, etc.), prevent acute liver failure, treat acute liver injury, increase hepatic macrophage numbers, and/or increase phagocytic activity when administered to a subject (e.g., human or animal subject). However, embodiments herein are not limited to any particular organ or mechanism of action, and an understanding of the mechanism of action is not necessary to practice such embodiments.

In some embodiments, provided herein are CPS1 proteins, polypeptides, and peptides (e.g., modified CPS1 polypeptides and peptides (e.g., having less than 100% sequence identity with SEQ ID NO: 1), and methods of use thereof for the treatment and/or prevention of acute internal tissue injury. In some embodiments, a CPS1 protein or polypeptide comprises at least 70% (e.g., >70%, >75%, >80%, <85%, >90%, >95%) sequence identity to SEQ ID NO: 1. In some embodiments, a CPS1 protein or polypeptide comprises at least 70% (e.g., >70%, >75%, >80%, <85%, >90%, >95%) sequence similarity (e.g., conservative or semiconservative) to SEQ ID NO: 1. In some embodiments, a CPS1 protein, polypeptide, or peptide comprises less than 100% sequence identity to SEQ ID NO: 1. In some embodiments, a CPS1 protein, polypeptide, or peptide is not an exact fragment of full-length CPS1 (e.g., 100% sequence identity to a portion of SEQ ID NO: 1). In other embodiments, a CPS1 peptide or polypeptide is a fragment of full-length CPS1 (SEQ ID NO: 1).

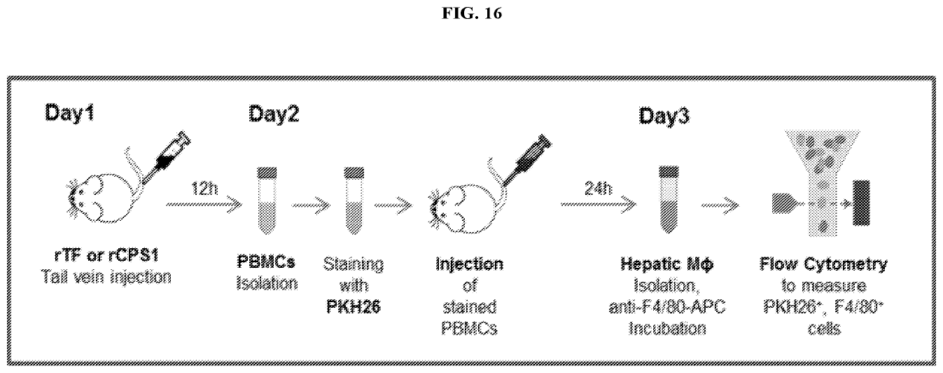

In some embodiments, a CPS1 polypeptide comprises one or more domains of wild-type CPS1 (e.g., 1 domain, 2 domains, 3 domains, 4 domains, 5 domains, 6 domains, 7 domains, or ranges therebetween). In some embodiments, a CPS1 polypeptide comprises at least 70% sequence identity (e.g., >70%, >75%, >80%, <85%, >90%, >95%) with one or more domains of wild-type CPS1 (e.g., 1 domain, 2 domains, 3 domains, 4 domains, 5 domains, 6 domains, 7 domains, or ranges therebetween). In some embodiments, a CPS1 polypeptide comprises at least 70% (e.g., >70%, >75%, >80%, <85%, >90%, >95%) sequence identity with the H domain of CPS1 (e.g., SEQ ID NO: 2). In some embodiments, a CPS1 polypeptide comprises at least 70% (e.g., >70%, >75%, >80%, <85%, >90%, >95%) sequence similarity (e.g., conservative or semiconservative) with the H domain of CPS1 (e.g., SEQ ID NO: 2). In some embodiments, a CPS1 polypeptide comprises at least 70% (e.g., >70%, >75%, >80%, <85%, >90%, >95%) sequence identity with the N-terminal domain of CPS1 (e.g., SEQ ID NO: 3). In some embodiments, a CPS1 polypeptide comprises at least 70% (e.g., >70%, >75%, >80%, <85%, >90%, >95%) sequence similarity (e.g., conservative or semiconservative) with the N-terminal domain of CPS1 (e.g., SEQ ID NO: 3). In some embodiments, a CPS1 polypeptide comprises at least 70% (e.g., >70%, >75%, >80%, <85%, >90%, >95%) sequence identity with the glutaminase-like domain of CPS1 (e.g., SEQ ID NO: 4). In some embodiments, a CPS1 polypeptide comprises at least 70% (e.g., >70%, >75%, >80%, <85%, >90%, >95%) sequence similarity (e.g., conservative or semiconservative) with the glutaminase-like domain of CPS1 (e.g., SEQ ID NO: 4). In some embodiments, a CPS1 polypeptide comprises at least 70% (e.g., >70%, >75%, >80%, <85%, >90%, >95%) sequence identity with the bicarbonate phosphorylation domain of CPS1 (e.g., SEQ ID NO: 5). In some embodiments, a CPS1 polypeptide comprises at least 70% (e.g., >70%, >75%, >80%, <85%, >90%, >95%) sequence similarity (e.g., conservative or semiconservative) with the bicarbonate phosphorylation domain of CPS1 (e.g., SEQ ID NO: 5). In some embodiments, a CPS1 polypeptide comprises at least 70% (e.g., >70%, >75%, >80%, <85%, >90%, >95%) sequence identity with the central domain of CPS1 (e.g., SEQ ID NO: 6). In some embodiments, a CPS1 polypeptide comprises at least 70% (e.g., >70%, >75%, >80%, <85%, >90%, >95%) sequence similarity (e.g., conservative or semiconservative) with the central domain of CPS1 (e.g., SEQ ID NO: 6). In some embodiments, a CPS1 polypeptide comprises at least 70% (e.g., >70%, >75%, >80%, <85%, >90%, >95%) sequence identity with the carbamate phosphorylation domain of CPS1 (e.g., SEQ ID NO: 7). In some embodiments, a CPS1 polypeptide comprises at least 70% (e.g., >70%, >75%, >80%, <85%, >90%, >95%) sequence similarity (e.g., conservative or semiconservative) with the carbamate phosphorylation domain of CPS1 (e.g., SEQ ID NO: 7). In some embodiments, a CPS1 polypeptide comprises at least 70% (e.g., >70%, >75%, >80%, <85%, >90%, >95%) sequence identity with the NAG-binding domain of CPS1 (e.g., SEQ ID NO: 8). In some embodiments, a CPS1 polypeptide comprises at least 70% (e.g., >70%, >75%, >80%, <85%, >90%, >95%) sequence similarity (e.g., conservative or semiconservative) with the NAG-binding domain of CPS1 (e.g., SEQ ID NO: 8). In some embodiments, a polypeptide comprises at least 70% (e.g., >70%, >75%, >80%, <85%, >90%, >95%) sequence identity (or similarity) with any suitable combination of the H domain, N-terminal domain, Glutaminase-like domain, bicarbonate phosphorylation domain, central domain, carbamate phosphorylation domain, and NAG-binding domain of CPS1. Exemplary truncated CPS1 polypeptides are depicted in FIG. 16. Embodiments herein are not limited to such exemplary polypeptides. In some embodiments, a CPS1 polypeptide comprises at least 70% (e.g., >70%, >75%, >80%, <85%, >90%, >95%) sequence identity (or similarity) with one of the truncated CPS1 polypeptides of FIG. 16.

In some embodiments, a CPS1 polypeptide comprises at least 70% (e.g., >70%, >75%, >80%, <85%, >90%, >95%) sequence identity (or similarity) with a CPS1 polypeptide lacking one or more domains of full-length CPS1. In some embodiments, a CPS1 polypeptide lacks any domain comprising greater than 25% (e.g., 25%, 50%, 75%, 90%, 100%, or ranges therebetween) sequence identity with one or more (e.g., 1, 2, 3, 4, 5, 6, or ranges therebetween) of the CPS1 H domain, N-terminal domain, Glutaminase-like domain, bicarbonate phosphorylation domain, central domain, carbamate phosphorylation domain, and NAG-binding domain of CPS1.

In some embodiments, a CPS1 polypeptide comprises 70% sequence identity to amino acids 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, 291-300, 301-310, 311-320, 321-330, 331-340, 341-350, 351-360, 361-370, 371-380, 381-390, 391-400, 401-410, 411-420, 421-430, 431-440, 441-450, 451-460, 461-470, 471-480, 481-490, 491-500, 501-510, 511-520, 521-530, 531-540, 541-550, 551-560, 561-570, 571-580, 581-590, 591-600, 601-610, 611-620, 621-630, 631-640, 641-650, 651-660, 661-670, 671-680, 681-690, 691-700, 701-710, 711-720, 721-730, 731-740, 741-750, 751-760, 761-770, 771-780, 781-790, 791-800, 801-810, 811-820, 821-830, 831-840, 841-850, 851-860, 861-870, 871-880, 881-890, 891-900, 901-910, 911-920, 921-930, 931-940, 941-950, 951-960, 961-970, 971-980, 981-990, 991-1000, 1001-1010, 1011-1020, 1021-1030, 1031-1040, 1041-1050, 1051-1060, 1061-1070, 1071-1080, 1081-1090, 1091-1100, 1101-1110, 1111-1120, 1121-1130, 1131-1140, 1141-1150, 1151-1160, 1161-1170, 1171-1180, 1181-1190, 1191-1200, 1201-1210, 1211-1220, 1221-1230, 1231-1240, 1241-1250, 1251-1260, 1261-1270, 1271-1280, 1281-1290, 1291-1300, 1301-1310, 1311-1320, 1321-1330, 1331-1340, 1341-1350, 1351-1360, 1361-1370, 1371-1380, 1381-1390, 1391-1400, 1401-1410, 1411-1420, 1421-1430, 1431-1440, 1441-1450, 1451-1460, 1461-1470, 1471-1480, 1481-1490, or 1491-1500 of CPS1 (SEQ ID NO: 1), or any combinations thereof.

In some embodiments, a CPS1 polypeptide lacks the enzymatic activity (e.g., the ability to transfer an ammonia molecule from glutamine or glutamate to a molecule of bicarbonate that has been phosphorylated by a molecule of ATP) of full-length, wild-type CPS1. In some embodiments, a CPS1 polypeptide exhibits the cytokine-like activity of full-length, wild-type CPS1. In some embodiments, a CPS1 polypeptide exhibits at least 50% of the cytokine-like activity of full-length, wild-type CPS1. In some embodiments, a CPS1 polypeptide exhibits enhanced (e.g., 2-fold, 3-fold, 4-fold, 5-fold, 10-fold, 100-fold, 1000-fold, or more, or ranges therebetween) cytokine-like activity of full-length, wild-type CPS1. In some embodiments, a CPS1 polypeptide exhibits enhanced solubility, biocompatibility, cell permeability, of other characteristics compared to wild-type CPS1.

In some embodiments, provided herein are peptides consisting of a fragment (e.g., a segment of 30 or fewer amino acids (e.g., 30, 28, 26, 24, 22, 20, 18, 16, 14, 12, 10, 8, or ranges therebetween)) of CPS1 or variants thereof (e.g., having at least 70% (e.g., 70%, 75%, 80%, 85%, 90%, 95%, 100%, or ranges therebetween) sequence identity with a corresponding segment of CPS1. In some embodiments, a CPS1 peptide comprises a portion of the H domain, N-terminal domain, Glutaminase-like domain, bicarbonate phosphorylation domain, central domain, carbamate phosphorylation domain, and NAG-binding domain of CPS1. In some embodiments, a CPS1 peptide comprises 70% sequence identity to amino acids 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, 291-300, 301-310, 311-320, 321-330, 331-340, 341-350, 351-360, 361-370, 371-380, 381-390, 391-400, 401-410, 411-420, 421-430, 431-440, 441-450, 451-460, 461-470, 471-480, 481-490, 491-500, 501-510, 511-520, 521-530, 531-540, 541-550, 551-560, 561-570, 571-580, 581-590, 591-600, 601-610, 611-620, 621-630, 631-640, 641-650, 651-660, 661-670, 671-680, 681-690, 691-700, 701-710, 711-720, 721-730, 731-740, 741-750, 751-760, 761-770, 771-780, 781-790, 791-800, 801-810, 811-820, 821-830, 831-840, 841-850, 851-860, 861-870, 871-880, 881-890, 891-900, 901-910, 911-920, 921-930, 931-940, 941-950, 951-960, 961-970, 971-980, 981-990, 991-1000, 1001-1010, 1011-1020, 1021-1030, 1031-1040, 1041-1050, 1051-1060, 1061-1070, 1071-1080, 1081-1090, 1091-1100, 1101-1110, 1111-1120, 1121-1130, 1131-1140, 1141-1150, 1151-1160, 1161-1170, 1171-1180, 1181-1190, 1191-1200, 1201-1210, 1211-1220, 1221-1230, 1231-1240, 1241-1250, 1251-1260, 1261-1270, 1271-1280, 1281-1290, 1291-1300, 1301-1310, 1311-1320, 1321-1330, 1331-1340, 1341-1350, 1351-1360, 1361-1370, 1371-1380, 1381-1390, 1391-1400, 1401-1410, 1411-1420, 1421-1430, 1431-1440, 1441-1450, 1451-1460, 1461-1470, 1471-1480, 1481-1490, or 1491-1500 of CPS1 (SEQ ID NO: 1), or any combinations thereof.

In some embodiments, a CPS1 peptide lacks the enzymatic activity (e.g., the ability to transfer an ammonia molecule from glutamine or glutamate to a molecule of bicarbonate that has been phosphorylated by a molecule of ATP) of full-length, wild-type CPS1. In some embodiments, a CPS1 peptide exhibits the cytokine-like activity of full-length, wild-type CPS1. In some embodiments, a CPS1 peptide exhibits at least 50% of the cytokine-like activity of full-length, wild-type CPS1. In some embodiments, a CPS1 polypeptide exhibits enhanced (e.g., 2-fold, 3-fold, 4-fold, 5-fold, 10-fold, 100-fold, 1000-fold, or more, or ranges therebetween) cytokine-like activity of full-length, wild-type CPS1. In some embodiments, a CPS1 peptide exhibits enhanced solubility, biocompatibility, cell permeability, of other characteristics compared to wild-type CPS1.

In some embodiments, provided herein are fusions of a CPS1 peptide or polypeptide described herein and a second peptide or polypeptide sequence. In some embodiments, the second peptide or polypeptide sequence is a functional peptide or polypeptide that facilitates delivery to tissues/cells, cell entry, bioavailability, permeability, solubility, etc. of the CPS1 peptide or polypeptide. In some embodiments, the second peptide or polypeptide is a therapeutic peptide or polypeptide that treats or prevents tissue damage by a similar or distinct mechanism from CPS1. In some embodiments, the second (functional) peptide or polypeptide segment comprises a signaling moiety, therapeutic moiety, localization moiety (e.g., cellular import signal, nuclear localization signal, etc.), detectable moiety (e.g., fluorescent moiety, contrast agent), or isolation/purification moiety (e.g., streptavidin, Hi S6, etc.). Such fusions may be expressed from a recombinant DNA which encodes the CPS1 polypeptide or peptide and the second peptide/polypeptide, or may be formed by chemical synthesis. For instance, the fusion may comprise the CPS1 polypeptide or peptide and an enzyme of interest, a luciferase, RNasin or RNase, and/or a channel protein (e.g., ion channel protein), a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, selectable marker protein, nucleic acid binding protein, extracellular matrix protein, secreted protein, receptor ligand, serum protein, a protein with reactive cysteines, a transporter protein, a targeting sequence (e.g., a myristylation sequence), a mitochondrial localization sequence, a plasma membrane penetrating peptide, or a nuclear localization sequence. The second peptide/polypeptide may be fused to the N-terminus and/or the C-terminus of the CPS1 polypeptide or peptide. In one embodiment, the fusion protein comprises a first peptide/polypeptide at the N-terminus and another (different) peptide/polypeptide at the C-terminus of the CPS1 polypeptide or peptide. Optionally, the elements in the fusion are separated by a connector sequence, e.g., preferably one having at least 2 amino acid residues, such as one having 13 and up to 40 or 50 amino acid residues. In some embodiments, the presence of a connector sequence in a fusion protein of the invention does not substantially alter the function of either element (e.g., the CPS1 polypeptide or peptide) in the fusion relative to the function of each individual element, likely due to the connector sequence providing flexibility (autonomy) for each element in the fusion. In certain embodiments, the connector sequence is a sequence recognized by an enzyme or is photocleavable. For example, the connector sequence may include a protease recognition site.

Embodiments are not limited to the specific sequences listed herein. In some embodiments, CPS1 peptides/polypeptides/fusions meeting limitations described herein (e.g., cytokine-like activity) and having substitutions not explicitly described are within the scope of embodiments here. In some embodiments, the peptides/polypeptides/fusions described herein are further modified (e.g., substitution, deletion, or addition of standard amino acids; chemical modification; etc.). Modifications that are understood in the field include N-terminal modification, C-terminal modification (which protects the peptide from proteolytic degradation), alkylation of amide groups, hydrocarbon “stapling” (e.g., to stabilize conformations). In some embodiments, the peptides/polypeptides described herein may be modified by conservative residue substitutions, for example, of the charged residues (K to R, R to K, D to E and E to D). Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The a-carbon of an amino acid may be mono- or dimethylated.

In some embodiments, CPS1 polypeptides, peptides, or fusions thereof are provided comprising: (i) one or more of the amino acid residues in the peptide are D-enantiomers, (ii) an N-terminally acetyl group, (iii) a deamidated C-terminal group, (iv) one or more unnatural amino acids, (v) one or more amino acid analogs, and/or (vi) one or more peptoid amino acids. In some embodiments, CPS1 polypeptides, peptides, or fusions thereof or an amino acid therein comprises a modification selected from the group consisting of phosphorylation, glycosylation, ubiquitination, S-nitrosylation, methylation, N-acetylation, lipidation, lipoylation, deimination, eliminylation, disulfide bridging, isoaspartate formation, racemization, glycation; carbamylation, carbonylation, isopeptide bond formation, sulfation, succinylation, S-sulfonylation, S-sulfinylation, S-sulfenylation, S-glutathionylation, pyroglutamate formation, propionylation, adenylylation, nucleotide addition, iodination, hydroxylation, malonylation, butyrylation, amidation, C-terminal amidation, de-amidation, alkylation, acylation, biotinylation, carbamylation, oxidation, and pegylation. In some embodiments, the peptide exhibits enhanced stability, solubility, cytokine-like activity, bioavalability, cell permeability, etc. relative to one of SEQ ID NOs: 1-8.

In some embodiments, any embodiments described herein may comprise mimetics corresponding to CPS1-derived polypeptides/peptides described herein and/or variants or fusions thereof, with various modifications that are understood in the field. In some embodiments, residues in the peptide sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally-occurring amino acids other than the standard 20 amino acids) are substituted in order to achieve desired properties.

In some embodiments, residues having a side chain that is positively charged under physiological conditions, or residues where a positively-charged side chain is desired, are substituted with a residue including, but not limited to: lysine, homolysine, 6-hydroxylysine, homoarginine, 2,4-diaminobutyric acid, 3-homoarginine, D-arginine, arginal (—COOH in arginine is replaced by —CHO), 2-amino-3-guanidinopropionic acid, nitroarginine (N(G)-nitroarginine), nitrosoarginine (N(G)-nitrosoarginine), methylarginine (N-methyl-arginine), ε-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine.

A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges. In some embodiments, residues having a side chain that is neutral/polar under physiological conditions, or residues where a neutral side chain is desired, are substituted with a residue including, but not limited to: asparagine, cysteine, glutamine, serine, threonine, tyrosine, citrulline, N-methylserine, homoserine, allo-threonine and 3,5-dinitro-tyrosine, and β-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, β-alanine, 1-aminocyclohexylcarboxylic acid, N-methylisoleucine, norleucine, norvaline, and N-methylvaline.

In some embodiments, peptide and polypeptides are isolated and/or purified (or substantially isolated and/or substantially purified). Accordingly, in such embodiments, peptides and/or polypeptides are provided in substantially isolated form. In some embodiments, peptides and/or polypeptides are isolated from other peptides and/or polypeptides as a result of solid phase peptide synthesis, for example. Alternatively, peptides and/or polypeptides can be substantially isolated from other proteins after cell lysis from recombinant production. Standard methods of protein purification (e.g., HPLC) can be employed to substantially purify peptides and/or polypeptides. In some embodiments, the present invention provides a preparation of peptides and/or polypeptides in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryprotectants (e.g., sugars such as trehalose), etc. The form of such preparations can be solutions, gels, etc. In some embodiments, peptides and/or polypeptides are prepared in lyophilized form. Moreover, such preparations can include other desired agents, such as small molecules or other peptides, polypeptides or proteins. Indeed, such a preparation comprising a mixture of different embodiments of the peptides and/or polypeptides described here may be provided.

In some embodiments, provided herein are peptidomimetic versions of the peptide sequences described herein or variants thereof. In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Cyclization (head-to-tail, head/tail-to-side-chain, and/or side-chain-to-side-chain) enhances peptide stability and permeability by introducing conformation constraint, thereby reducing peptide flexibility, and a cyclic enkephalin analog is highly resistant to enzymatic degradation. Chapter 14 of “Drug Design and Development”, Krogsgaard, Larsen, Liljefors and Madsen (Eds) 1996, Horwood Acad. Publishers provides a general discussion of techniques for the design and synthesis of peptidomimetics and is herein incorporated by reference in its entirety. Suitable amide bond surrogates include, but are not limited to: N-alkylation (Schmidt, R. et al., Int. J. Peptide Protein Res., 1995, 46, 47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. O. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methyleneamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J. Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g. borane or a hydride reagent such as lithium aluminum-hydride); such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142; herein incorporated by reference in its entirety.

In some embodiments, provided herein are pharmaceutical compositions comprising one the CPS1 polypeptides, peptides, fusions or variants thereof and a pharmaceutically acceptable carrier. Any carrier which can supply an active peptide or polypeptide (e.g., without destroying the peptide or polypeptide within the carrier) is a suitable carrier, and such carriers are well known in the art. In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intracisternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

In some embodiments, provided herein are methods for treating patients suffering from acute tissue injury, and/or in need of treatment (or preventative therapy). In some embodiments, a pharmaceutical composition comprising a CPS1 polypeptide or peptide (or fusions or variants thereof) is delivered to such a patient in an amount and at a location sufficient to treat/prevent the condition. In some embodiments, peptides and/or polypeptides (or pharmaceutical composition comprising such) are delivered to the patient systemically or locally, and it will be within the ordinary skill of the medical professional treating such patient to ascertain the most appropriate delivery route, time course, and dosage for treatment. It will be appreciated that application methods of treating a patient most preferably substantially alleviates or even eliminates such symptoms; however, as with many medical treatments, application of the inventive method is deemed successful if, during, following, or otherwise as a result of the inventive method, the symptoms of the disease or disorder in the patient subside to an ascertainable degree. In some embodiments, the success of treatment or prevention is determined on a population basis, rather than based on a single patient (e.g., did the overall risk for a particular population of disease or tissue injury decrease?).

In some embodiments, the compositions and methods herein find use in the treatment or prevention of tissue injury, for the restoration or rejuvenation of tissue in a subject in need thereof. In some embodiments, the tissue or cells therein may be neuronal, eye (e.g., retinal), adipose, connective, hair, teeth, mucosal, pancreas, endocrine, ear, epithelial, skin, muscle, heart, lung, liver, kidney, intestine, adrenal (e.g., adrenal cortex, adrenal medulla), capillary, endothelial, testes, ovary, bone, skin, or endometrial cells or tissue.

In some embodiments, a cell, tissue, or organ for which the compositions and methods herein are used to prevent/treat injury are those that been exposed to at least one condition (e.g., disease state) adverse to the viability of the cell, tissue, or organ. In accordance with this embodiment, the compositions and methods herein provide for treatment/prevention/restoration of tissue and/or tissue function in a subject in need thereof before, during or after such conditions arise. Such conditions include traumatic in situ hypoxia or metabolic dysfunction, surgically-induced in situ hypoxia or metabolic dysfunction, or in situ toxin exposure, a seizure disorder, multiple sclerosis, stroke, hypotension, cardiac arrest, ischemia, myocardial infarction, inflammation, age-related loss of cognitive function, radiation damage, cerebral palsy, neurodegenerative disease, Alzheimer's disease, Parkinson's disease, mitochondrial disease, AIDS dementia, memory loss, amyotrophic lateral sclerosis, alcoholism, mood disorder, anxiety disorder, attention deficit disorder, autism, Creutzfeld-Jakob disease, brain or spinal cord trauma or ischemia, heart-lung bypass, heart failure, macular degeneration, diabetic neuropathy, diabetic retinopathy, hepatitis, pancreatitis, glaucoma, retinal ischemia, retinal trauma, cardiovascular disease, cardiopulmonary disease, respiratory disease, kidney disease, disease of the urinary system, disease of the reproductive system, bone disease, skin disease, connective tissue disease, gastrointestinal disease, endocrine abnormality, metabolic abnormality, a disease or disorder of the central or peripheral nervous system, etc.

A pharmaceutical composition may be administered in the form which is formulated with a pharmaceutically acceptable carrier and optional excipients, adjuvants, etc. in accordance with good pharmaceutical practice. The CPS1 polypepitde/peptide (or fusions or variants thereof) pharmaceutical composition may be in the form of a solid, semi-solid or liquid dosage form: such as powder, solution, elixir, syrup, suspension, cream, drops, paste and spray. As those skilled in the art would recognize, depending on the chosen route of administration (e.g. pill, injection, etc.), the composition form is determined. In general, it is preferred to use a unit dosage form in order to achieve an easy and accurate administration of the active pharmaceutical peptide or polypeptide. In general, the therapeutically effective pharmaceutical compound is present in such a dosage form at a concentration level ranging from about 0.5% to about 99% by weight of the total composition, e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, pharmaceutical compositions of CPS1 polypeptides/peptides described herein (or fusions or variants thereof) are provided in a unit dosage form for administration to a subject, comprising one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

In various embodiments, the polypeptides/peptides disclosed herein are derivatized by conjugation to one or more polymers or small molecule substituents.

In certain of these embodiments, the CPS1 polypeptides/peptides described herein (or fusions or variants thereof) are derivatized by coupling to polyethylene glycol (PEG). Coupling may be performed using known processes. See, Int. J. Hematology, 68:1 (1998); Bioconjugate Chem., 6:150 (1995); and Crit. Rev. Therap. Drug Carrier Sys., 9:249 (1992) all of which are incorporated herein by reference in their entirety. Those skilled in the art, therefore, will be able to utilize such well-known techniques for linking one or more polyethylene glycol polymers to the peptides and polypeptides described herein. Suitable polyethylene glycol polymers typically are commercially available or may be made by techniques well known to those skilled in the art. The polyethylene glycol polymers preferably have molecular weights between 500 and 20,000 and may be branched or straight chain polymers.

The attachment of a PEG to a peptide or polypeptide described herein can be accomplished by coupling to amino, carboxyl or thiol groups. These groups will typically be the N- and C-termini and on the side chains of such naturally occurring amino acids as lysine, aspartic acid, glutamic acid and cysteine. Since the peptides and polypeptides of the present disclosure can be prepared by solid phase peptide chemistry techniques, a variety of moieties containing diamino and dicarboxylic groups with orthogonal protecting groups can be introduced for conjugation to PEG.

The present disclosure also provides for conjugation of CPS1 polypeptides/peptides described herein (or fusions or variants thereof) to one or more polymers other than polyethylene glycol.

In some embodiments, CPS1 polypeptides/peptides described herein (or fusions or variants thereof) are derivatized by conjugation or linkage to, or attachment of, polyamino acids (e.g., poly-his, poly-arg, poly-lys, etc.) and/or fatty acid chains of various lengths to the N- or C-terminus or amino acid residue side chains. In certain embodiments, the peptides and polypeptides described herein are derivatized by the addition of polyamide chains, particularly polyamide chains of precise lengths, as described in U.S. Pat. No. 6,552,167, which is incorporated by reference in its entirety. In yet other embodiments, the peptides and polypeptides are modified by the addition of alkylPEG moieties as described in U.S. Pat. Nos. 5,359,030 and 5,681,811, which are incorporated by reference in their entireties.

In select embodiments, CPS1 polypeptides/peptides described herein (or fusions or variants thereof) are derivatized by conjugation to polymers that include albumin and gelatin. See, Gombotz and Pettit, Bioconjugate Chem., 6:332-351, 1995, which is incorporated herein by reference in its entirety.

In further embodiments, CPS1 polypeptides/peptides described herein (or fusions or variants thereof) are conjugated or fused to immunoglobulins or immunoglobulin fragments, such as antibody Fc regions.

In some embodiments, the pharmaceutical compositions described herein (e.g., comprising CPS1 polypeptides/peptides described herein (or fusions or variants thereof) find use in the treatment and/or prevention of internal tissue injury, acute tissue injury, and related conditions. In some embodiments, the compositions are administered to a subject. In certain embodiments, the patient is an adult. In other embodiments, the patient is a child.

In various embodiments, CPS1 polypeptides/peptides described herein (or fusions or variants thereof) are administered in an amount, on a schedule, and for a duration sufficient to decrease triglyceride levels by at least 5%, 10%, 15%, 20% or 25% or more as compared to levels just prior to initiation of treatment. In some embodiments, CPS1 polypeptides/peptides described herein (or fusions or variants thereof) are administered in an amount, on a dosage schedule, and for a duration sufficient to increases hepatic macrophage numbers by at least 10%, 15%, 20%, 25%, 30%, 35%, 40%, 45% 50%, 60%, 70%, 80%, 90%, 100%. In some embodiments, CPS1 polypeptides/peptides described herein (or fusions or variants thereof) are administered in an amount, on a dosage schedule, and for a duration sufficient to increases phagocytic activity by at least 10%, 15%, 20%, 25%, 30%, 35%, 40%, 45% 50%, 60%, 70%, 80%, 90%, 100%.

In certain embodiments, CPS1 polypeptides/peptides described herein (or fusions or variants thereof) are administered in an amount, expressed as a daily equivalent dose regardless of dosing frequency, of 50 micrograms (“mcg”) per day, 60 mcg per day, 70 mcg per day, 75 mcg per day, 100 mcg per day, 150 mcg per day, 200 mcg per day, or 250 mcg per day. In some embodiments, CPS1 polypeptides/peptides described herein (or fusions or variants thereof) are administered in an amount of 500 mcg per day, 750 mcg per day, or 1 milligram (“mg”) per day. In yet further embodiments, CPS1 polypeptides/peptides described herein (or fusions or variants thereof) are administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day.

In various embodiments CPS1 polypeptides/peptides described herein (or fusions or variants thereof) are administered on a monthly, biweekly, weekly, daily (“QD”), or twice a day (“BID”) dosage schedule. In typical embodiments, the peptide/polypeptide is administered for at least 3 months, at least 6 months, at least 12 months, or more. In some embodiments, CPS1 polypeptides/peptides described herein (or fusions or variants thereof) are administered for at least 18 months, 2 years, 3 years, or more.

### EXPERIMENTAL

### Materials and Methods

### Mouse Experiments

Mouse procedures were approved by the University Committee on Use and Care of Animals at the University of Michigan. FVB/N mice were obtained from Jackson Laboratory and were used in experiments. For liver injury, overnight fasted (APAP) or fed (FL) 8 week-old mice were intraperitonealy (ip) injected with APAP (450 mg/kg for females; 350 mg/kg for males) or FL (0.15 mg/kg). After 4 h or at the indicated times, mice were euthanized by CO₂ inhalation, followed by blood collection (by heart puncture) for ALT measurement (Pointe Scientific, Inc.). The livers were then removed and divided into pieces for hematoxylin and eosin staining or snap frozen in liquid nitrogen for subsequent biochemical analysis. For the recombinant protein injection experiments, mice were placed in a restrainer that allowed placing the tail in warm water (˜32° C.) for a few seconds to stimulate tail vein dilation, followed by slow injection of purified rTF or rCPS1 [25 μg or 50 μg, respectively, providing similar moles of protein (rTF=78 kD, rCPS1=160 kD)] in 100 μl of buffer containing 50 mM NaH₂PO₄, 500 mM NaCl, 200 mM imidazole, 10% glycerol, 1 mM DTT, pH 8). At 24 h post injection, mice were given (ip) FL or APAP to induce liver injury, or were euthanized for further experiments. For the therapeutic approach, mice were injected ip with APAP first, followed by sampling of blood from the tail vein (˜40 μl/collection) at 3 hour intervals to measure ALT changes in the same animals over time. Then, recombinant proteins were injected via tail vein, followed by additional blood collection every 3 h until 12 h after the first injection. For the clodronate-induced macrophage depletion experiment, 200 μl of clodronate liposomes (Liposoma) or PBS (as a control) were administered per mouse intraperitonealy 48 h prior to rTF or rCPS1 administration.

### Primary Cell Isolation and Culture

Hepatocyte isolation was performed as described in Weerasinghe et al. (2014) Am J Physiol Gastrointest Liver Physiol 307:G355-364.; herein incorporated by reference in its entirety. The liver was perfused with perfusion medium through the portal vein for 2 min (3 ml/min flow rate), followed by perfusion of 20 ml of digestion medium containing 3000 U collagenase type 2 (Worthington) at the same flow rate. Isolated cells were purified by Percoll (Sigma, 30% in PBS) gradient centrifugation. The washed pellet was suspended in culture medium and plated [2×10⁵ cells/ml on collagen-I-coated 6-well plates (BD BioCoat)] for subsequent analysis. After 2 h or 16 h post-plating, the culture medium was exchanged with Williams' Media E (Invitrogen) and treated with saline or FL (0.5 μg/ml) with or without combination of other reagents as indicated.

PBMCs were isolated from whole blood using Histopaque-1077 (for human cells) or Histopaque-1083 (for mouse cells) gradient centrifugation. Existing human blood samples that would otherwise be discarded were obtained the Hematology Laboratory at the University of Michigan Medical Center, via an approved Human Subjects protocol. Erythrocytes were removed using RBC lysis buffer (Sigma) followed by washing with HBSS. The cells were plated and monocytes were allowed to attach on non-coated 12-well plates (Thermo Fischer Scientific). After 2 h, the non-attached cells were removed and the adhered cells (Mϕ) were washed twice with PBS and cultured in RPMI-1640 medium and 10% fetal bovine serum.

For isolation of hepatic Mϕ, mouse liver was perfused with 10 ml of PBS and minced. Then, the liver fragments were incubated with RPMI-1640 medium containing 0.1% collagenase type 4 (Worthington) for 30 min at 37° C., followed by filtration through a 70 μm mesh (Thermo Fisher Scientific). After two washes (with pelleting at 300×g), the cells were pelleted (50×g, 3 min) and the supernatant was transferred to a new tube which was centrifuged (300×g, 5 min). The last pellet containing non-parenchymal cells and endothelial cells were plated and hepatic Mϕ were allowed to attach on non-coated 12-well plates. After 2 h, the attached cells were washed twice with PBS and cultured in complete RPMI-1640 medium. For the co-culture experiments, naïve Kupffer cells and PBMCs were isolated separately as indicated above and plated on the lower and upper wells of transwell plates (0.4 μm pore, Sigma), respectively, followed by 24 h culture.

Aortic endothelial cells were isolated as described in Kobayashi M, et al. (2005) J Atheroscler Thromb. 12, 138-142.; herein incorporated by reference in its entirety. Mouse aorta was dissected from the aortic arch to the abdominal aorta and immersed in 20% FBS-DMEM containing 1,000 U/ml of heparin (Sagent Pharmaceuticals) after trimming of fat and connective tissue under a microscope. The lumen was rinsed with DMEM through a catheter inserted into the proximal aorta, then filled and incubated with a collagenase type 2 solution (2 mg/ml in DMEM; 45 min, 37° C.). The detached endothelial cells were collected by flushing then pelleting (300×g, 5 min), and the pellet was resuspended and plated on collagen-I-coated 6-well plates. After 2 h, the attached cells were rinsed while on the dish with PBS to remove smooth muscle cells, then cultured in complete EGM-2 medium (Lonza).

Bone marrow cells were isolated as described in Amend S R, et al. (2016) J Vis Exp. 110.; herein incorporated by reference in its entirety. Mouse femur and tibia were isolated and any additional muscle or connective tissues attached were removed. After removal of the condyles using a scissors to expose the metaphysis, the bones were placed into a 0.5 ml microcentrifuge tube punched at the bottom, and the tubes were nested in an intact 1.5 ml centrifuge tube, followed by centrifugation at 10,000×g for 15 sec. The collected bone marrow was subjected to quantitative RT-PCR.

### Microarray Analysis

Total RNA of hepatic Mϕ from mice, injected with rCPS1 or rTF (n=4/group) followed by APAP administration, was converted to cDNA and biotinylated as recommended by Affymetrix GeneChip™ WT PLUS, starting with 400 ng total RNA. Biotinylated cDNAs were hybridized to the Mouse Gene 2.1 ST array using the GeneTitan Multi-Channel system (software version 4.3.0.1592). The probesets that had a variance less than 0.05 were filtered out and probesets with a fold change of 1.5 or greater were selected. p-values were adjusted for multiple comparisons using false discovery rate (FDR). The open access Gene Expression Omnibus series accession number is GSE122879.

### Flow Cytometry

For experiments testing circulating monocyte homing to the liver, 8-week old male FVB/N mice were administered with rTF or rCPS1 as indicated above. After 12 h, PBMCs were isolated from them, stained with PKH26 (Sigma) for 2 min, followed by washing four times, according to the manufacture recommendation, then injection into mice via tail vein (2×10⁵ cells in 150 μl of PBS). At 24 h post injection, hepatic Mϕ were isolated and incubated with APC-labeled F4/80 antibodies for 20 min on ice in the dark, followed by washing 3× (300×g for 5 min). Single color controls were included for gating purposes. The cells were analyzed on a Beckman Coulter MoFlo Astrios at the University of Michigan Flow Cytometry core facility.

### Isolation of EVs, Sucrose Gradient Separation, and Biochemical Analysis

For collecting EVs, hepatocyte culture media was centrifuged sequentially using low speed (300×g for 10 min, then 2,000×g for 20 min) then ultracentrifuged (100,000×g, 90 min), followed by washing in PBS and pelleting using the same speed. Serum or bile samples were also processed similarly after dilution with equal volume of PBS. For separating EVs based on their size, cell-depleted culture media (after the 300×g for 10 min, then 2,000×g for 20 min spins) were then serially centrifuged at 10,000×g for 30 min (which typically pellets apoptotic bodies), 20,000×g for 30 min (pellets microvesicles) and 100,000×g for 90 min (pellets exosomes). Each pellet was washed with PBS and re-spun at the same speed and resuspended in PBS or SDS sample buffer for subsequent analysis. For sucrose gradient centrifugation, samples were loaded on top of a sucrose gradient that include 2.5 M (2 ml), 2 M (5 ml), and 0.25 M (5 ml) sucrose solutions in 20 mM HEPES buffer, and sedimented (210,000×g, 14 h, 4° C.). Fractions (1 ml each) were collected then diluted 3-fold and repelleted (10,000×g, 1 h). The pellets were resuspended in SDS sample buffer followed by immunoblotting.

For immunoblot analysis, cultured cells and liver tissues were lysed in 2× Tris-glycine SDS sample buffer. Sera or bile were also mixed with Tris-glycine SDS sample buffer before analysis. Proteins were subjected to SDS-polyacrylamide gel electrophoresis, then stained with GelCode Blue Stain Reagent (Thermo Fisher Scientific) or transferred to polyvinylidene difluoride membrane for blotting. For dot blotting, isolated EVs were spotted on a nitrocellulose membrane using Minifold 1 (Schleicher & Schuell), then, incubated with anti-CPS1 in the presence or absence of 0.1% Tween-20. All antibody information is included in Table 1. Quantitative RT-PCR. RNA was extracted in TRIzol (Invitrogen) and isolated according to the manufacture's protocol, then 1 μg of RNA was reverse transcribed to cDNA using TaqMan reverse transcriptase kit (Applied Biosystems). Quantitative PCR was done using Brilliant SYBR Green Master Mix (Bio-Rad) and Eppendorf MasterCycler RealPlex (Thermo Fisher Scientific). Primer information is included in Table 2.

TABLE 1
Antibody list

| | | | | Size |
|---|---|---|---|---|
| Antigen | Clone ID | Manufacturer | Application | (kD) |
| Primary Antibodies | | | | |
| CPS1 | ab45956 | Abcam | WB, IF | 160 |
| | ab129076 | Abcam | IP | |
| | ab3682 | Abcam | Immunogold-EM | N.A. |
| HMGB1 | ab79823 | Abcam | WB | 25 |
| LDH | LS-B5974 | LSBio | WB | 37 |
| Cyt c | ab133504 | Abcam | WB | 14 |
| PDH | 66119-1-Ig | Porteintech | WB | 43 |
| Tom20 | sc-17764 | Santa Cruz Biotechnology | WB, IF | 20 |
| CD9 | sc-13118 | Santa Cruz Biotechnology | WB | 24 |
| TSG101 | ab83 | Abcam | WB | 47 |
| cleaved caspase 3 | 9664 | Cell Signaling Technology | WB | 17, 19 |
| cleaved caspase 7 | 9491 | Cell Signaling Technology | WB | 20 |
| Actin | MA5-11869 | Thermo Fisher Scientific | WB | 43 |
| GRP78 | 3177 | Cell Signaling Technology | WB | 78 |
| Bax | MS-1335-P | Neomarkers | WB | 21 |
| Transferrin | sc-33731 | Santa Cruz Biotechnology | WB, IF | 79 |
| Amylase | sc-166349 | Santa Cruz Biotechnology | WB | 53 |
| Albumin | sc-46291 | Santa Cruz Biotechnology | WB | 66 |
| OTC | ab203859 | Abcam | WB | 40 |
| ASS1 | sc-365475 | Santa Cruz Biotechnology | WB | 47 |
| ASL | sc-166787 | Santa Cruz Biotechnology | WB | 51 |
| Arginase 1 | sc-271430 | Santa Cruz Biotechnology | WB | 35 |
| Chymotrypsin | 20-CR79 | Filzgerald | WB | 26 |
| Chymase | sc-59586 | Santa Cruz Biotechnology | WB | 30 |
| EF-Tu | sc-393924 | Santa Cruz Biotechnology | WB | 50 |
| AASS | sc-390511 | Santa Cruz Biotechnology | WB | 120 |
| PCCA | sc-374341 | Santa Cruz Biotechnology | WB | 70 |
| Elastase | sc-54187 | Santa Cruz Biotechnology | WB | 28 |
| Vimentin | V 6630 | Sigma | WB, IF | 58 |
| His | sc-8036 | Santa Cruz Biotechnology | WB | N.A. |
| cleaved K18 | | Lab-made | WB | 25 |
| Ki-67 | ab16667 | Abcam | WB, IF | 395 |
| p-Rb (S780) | 8180P | Cell Signaling Technology | WB | 110 |
| F4/80 | 14-4801-85 | eBioscience | IF | N.A. |
| Secondary Antibodies | | | | |
| Rabbit IgG-HRP | A6154 | Sigma | WB | N.A. |
| Mouse IgG-HRP | A4416 | Sigma | WB | N.A. |
| Goat IgG-HRP | A5420 | Sigma | WB | N.A. |
| Rabbit Alexa Fluor 488 | A11008 | Invitrogen | IF | N.A. |
| Rabbit Alexa Fluor 647 | A21244 | Invitrogen | IF | N.A. |
| Mouse Alexa Fluor 680 | A21057 | Invitrogen | IF | N.A. |
| Rabbit IgG-10 nm gold | 25109 | Electron Microscopy | Immunogold-EM | N.A. |
| conjugated | | Sciences | | |

TABLE 2
Q-PCR primer list

| Gene | Orientation | Sequence (5′→3′) |
|---|---|---|
| mouse Nos2 | F | GTTCTCAGCCCAACAATACAAGA |
| | R | GTGGACGSGTCGATGTCAC |
| mouse Cxcl10 | F | CCAAGTGCTGCCGTCATTTTC |
| | R | GGCTCGCAGGGATGATTTCAA |
| mouse II6 | F | TAGTCCTTCCTACCCCAATTTCC |
| | R | TTGGTCCTTAGCCACTCCTTC |
| mouse Arg1 | F | CTCCAAGCCAAAGTCCTTAGAG |
| | R | AGGAGCTGTCATTAGGGACATC |
| mouse Ccl22 | F | AGGTCCCTATGGTGCCAATGT |
| | R | CGGCAGGATTTTGAGGTCCA |
| mouse II10 | F | GCTCTTACTGACTGGCATGAG |
| | R | CGCAGCTCTAGGAGCATGTG |
| mouse Ccr1 | F | TGGGTGAACGGTTCTGGAAG |
| | R | GGTCCTTTCTAGTTGGTCCACA |
| mouse Cxcr2 | F | ATGCCCTCTATTCTGCCAGAT |
| | R | GTGCTCCSGTTGTATAAGATGAC |
| mouse Cps1 | F | ACATGGTGACCAAGATTCCTCG |
| | R | TTCCTCAAAGGTGCGACCAAT |
| mouse 18s | F | AAACGGCTACCACATCCAAG |
| | R | CCTCAAATGGATCCTCGTTA |
| human CD64 | F | GCATGGGAAAGCATCGCTAC |
| | R | GCAAGAGCAACTTTGTTTCACA |
| human CXCL10 | F | GTGGCATTCAAGGAGTACCTC |
| | R | GTGGCATTCAAGGAGTACCTC |
| human IL6 | F | CCTGAACCTTCCAAAGATGGC |
| | R | TTCACCAGGCAAGTCTCCTCA |
| human MRC1 | F | GGGTTGCTATCACTCTCTATGC |
| | R | TTTCTTGTCTGTTGCCGTAGTT |
| human IL10 | F | TCAAGGCGCATGTGAACTCC |
| | R | GATGTCAAACTCACTCATGGCT |
| human CCL22 | F | ATTACGTCCGTTACCGTCTGC |
| | R | TCCCTGAAGGTTAGCAACACC |
| human ACTB | F | CATGTACGTTGCTATCCAGGC |
| | R | CTCCTTAATGTCACGCACGAT |

### Bile Collection

For mouse bile collection, mice were anesthetized with isoflurane and a PE-08 catheter was inserted into the common bile duct using a dissecting microscope and glued in place. The mice were maintained under anesthesia and placed under a warming lamp, and bile was collected for 2 hours in microcentrifuge tubes containing Protease inhibitor cocktail (Invitrogen) at 20-minute intervals.

For human bile collection, bile samples were collected from patients undergoing endoscopic retrograde cholangiopancreatography for indicated clinical reasons, and carried out at the University of Michigan Medical Center under an institutional review board IRB-approved protocol. For Western blot analysis of human bile samples, 1 ml of human bile was precipitated with six volumes of −20° C. acetone (overnight, −80° C.) to remove interfering substances, followed by centrifugation. The pellet was dissolved in 200 μl of Tris-glycine SDS-containing sample buffer and 10 μl of each samples was subjected to SDS-PAGE separation.

### Protein Identification by LC-Tandem MS

Mouse bile samples collected from the common bile duct (CBD) or the gallbladder (GB) were analyzed by mass spectrometry. CBD and GB bile samples were combined from 3 mice and 20 μl of bile was incubated with 6 volumes of cold acetone (−80° C., overnight). The air-dried pellet after 16,000×g spin (4° C., 10 min) was dissolved in 40 μl of 50 mM Hepes/8 M urea. The bile protein extracts (10 μl of the 40 μl) were separated by SDS-PAGE and stained with GelCode Blue Stain Reagent (Thermo Fisher Scientific). Each lane was cut into 13 equal sized slices and analyzed by the Proteomics Resource Facility at the University of Michigan using an LC-MS based approach. Briefly, gel slices were destained with 30% methanol for 4 h. Upon reduction (10 mM DTT) and alkylation (65 mM 2-chloroacetamide) of the cysteines, proteins were digested overnight with 500 ng of sequencing grade modified trypsin (Promega). The resulting peptides were resolved on a nano-capillary reverse phase column (Acclaim PepMap C18, 2 micron, 50 cm, Thermo Fisher Scientific) using 0.1% formic acid/acetonitrile gradient at 300 nl/min (2-25% acetonitrile in 35 min; 25-50% acetonitrile in 20 min followed by a 90% acetonitrile wash for 5 min and a further 30 min re-equilibration with 2% acetonitrile) and directly introduced in to Q Exactive HF mass spectrometer (Thermo Fisher Scientific, San Jose Calif.). MS1 scans were acquired at 60K resolution. Data-dependent high-energy C-trap dissociation MS/MS spectra were acquired with top speed option (3 sec) following each MS1 scan (relative CE˜28%). Proteins were identified by searching the data against Mus musculus database (UniProtKB, v2016-4-13) and Proteome Discoverer (v2.1, Thermo Fisher Scientific). Search parameters included MS1 mass tolerance of 10 ppm and fragment tolerance of 0.1 Da; two missed cleavages were allowed; carbamidimethylation of cysteine was considered fixed modification and oxidation of methionine, deamidation of asparagine and glutamine, phosphorylation of serine, threonine, tyrosine were considered as potential modifications. False discovery rate (FDR) was determined using Percolator and proteins/peptides with a FDR of <1% were retained.

### Nanoparticle Tracking Analysis (NTA)

For measuring EV size and concentration, EV samples were diluted with PBS to be in a range between 20 and 80 particles per frame then analyzed using scatter mode of the NanoSight NS300 instrument (at 23.3° C.; syringe pump at 20 μl/min). Five videos of 1 min each documenting Brownian motion of nanoparticles were recorded, followed by analysis using NanoSight software. To analyze the GFP-containing EVs, samples were analyzed under the fluorescence mode with a 488 nm wavelength laser.

### Immunofluorescence Staining and Immunogold Staining Electron Microscopy.

5 μm-thick paraffin sections of liver were deparaffinized with xylene and rehydrated through a series of graded ethanol. For F4/80 staining, antigen retrieval was performed in boiling citrate buffer (10 mM sodium citrate, 0.05% Tween-20, pH 6). For Ki-67 staining, protease K-mediated antigen retrieval was performed (20 μg/ml of protease K in Tris-EDTA buffer, pH 8). The sections were blocked with 10% goat serum in PBS and incubated with primary antibodies (1:50 for F4/80 and 1:500 for Ki-67), followed by incubation with fluorphore-conjugated secondary antibody (1 h, 22° C.). Washed sections were mounted using ProLong Gold Antifade Mountant with DAPI (Thermo Fisher Scientific), and five random images per sample were acquired using a Zeiss Axio Imager 2 microscope followed by counting of positive stained cells. Expression and purification of recombinant proteins. Human CPS1 clone (ID: HsCD00342929) was obtained from DF/HCC DNA Resource Core at Harvard Medical School. To generate a His-tagged recombinant CPS1, pET-28a-hCPS1 was constructed by ligation of the PCR-generated hCPS1 ORF lacking the N-terminal mitochondrial targeting sequence (117 bp) into the EcoRI-XhoI sites of the pET-28a vector in frame with N-terminal or C-terminal His tag. Then, pFastBac-hCPS1 with His-tag was generated using the Gibson assembly cloning method. As a control, pFastBac-hTF mutant with His-tag was generated using Gibson assembly from the clone obtained from Addgene (pNUT N6His Y95F/Y188F/Y426F/Y517F hTFNG, N-His tagged nonglycosylated human serum transferrin, which is unable to bind iron in the N-lobe). For an enzymatically inactive CPS1, a T471N mutation was generated using QuikChange Lightning Multi Site-Directed Mutagenesis Kit (Agilent) Pekkala S et al. (2010) Hum Mutat 31:801-808.; herein incorporated by reference in its entirety). All clones were sequenced in their entirety to confirm the predicted sequences and lack of any inadvertent additional mutation. To produce Baculovirus expressing rCPS1 wild type, rCPS1 T471N or rTF, Bac-to-Bac Baculovirus Expression System (Invitrogen). The recombinant bacmids confirmed by PCR, were transfected into Sf9 insect cells to produce recombinant baculovirus, using Cellfectin II/unsupplemented Grace medium, followed by media change at 5 h post-transfection. After additional culture in 51900 medium (72 h, 27° C.), the culture was centrifuged at 500×g for 5 min, and the supernatant was used as a P1 viral stock. To express recombinant proteins, Sf9 cells (2×10⁶ cells/ml) were infected with amplified P2 or P3 stock viruses at MOI 1 then harvested at 72 h post-infection.

### Expression and Purification of the Recombinant Proteins (Diez-Fernandez C, et al. (2014) Mol Genet Metab 112:123-132.; Herein Incorporated by Reference in its Entirety)

Insect cell expression and purification of human CPS1 were performed as described (53) with slight modification. Human CPS1 clone (ID: HsCD00342929) was obtained from DF/HCC DNA Resource Core at Harvard Medical School. To generate a His-tagged recombinant CPS1, pET-28a-hCPS1 was constructed by ligation of the PCR-generated hCPS1 ORF lacking the N-terminal mitochondrial targeting sequence (117 bp) into the EcoRI-XhoI sites of the pET-28a vector in frame with N-terminal or C-terminal His tag. Then, pFastBac-hCPS1 with His-tag was generated using the Gibson assembly cloning method. As a control, pFastBac-hTF mutant with His-tag was generated using Gibson assembly from the clone obtained from Addgene (pNUT N6His Y95F/Y188F/Y426F/Y517F hTFNG, N-His tagged nonglycosylated human serum transferrin, which is unable to bind iron in the N-lobe). For an enzymatically inactive CPS1, a T471N mutation was generated using QuikChange Lightning Multi Site-Directed Mutagenesis Kit (Agilent) according to the manufacture's protocol (Pekkala S et al. (2010) Hum Mutat 31:801-808.; herein incorporated by reference in its entirety). All clones were sequenced in their entirety to confirm the predicted sequences and lack of any inadvertent additional mutation. To produce Baculovirus expressing rCPS1 wild type, rCPS1 T471N or rTF, Bac-to-Bac Baculovirus Expression System (Invitrogen) was used per manufacturer's instructions. Briefly, the recombinant bacmids confirmed by PCR, were transfected into Sf9 insect cells to produce recombinant baculovirus, using Cellfectin II/unsupplemented Grace medium, followed by media change at 5 h post-transfection. After additional culture in Sf900 medium (72 h, 27° C.), the culture was centrifuged (500×g, 5 min), and the supernatant was used as a P1 viral stock. To express recombinant proteins, Sf9 cells (2×106 cells/ml) were infected with amplified P2 or P3 stock viruses at MOI 1 then harvested at 72 h post-infection.

The infected insect cells from 1 L culture was suspended in 50 ml of a lysis solution [50 mM glycyl-glycine, pH 7.4, 10% glycerol, 20 mM KCl, 0.1% Triton X-100, 1 mM DTT, 1 mM PMSF, and 1% His protease inhibitor cocktail (Sigma)] and lysed by freezing-thawing three times. The viscous lysate was passed through an 18-gauge syringe needle to shear nuclear DNA, followed by centrifugation (16,000×g, 10 min), and the supernatants were subjected to purification using HisPur Ni-NTA resin (Thermo Fisher Scientific). 10 ml of lysates were incubated with 2 ml of washed resin in a total of 50 ml of binding buffer (50 mM NaH₂PO₄, 500 mM NaCl, 5 mM imidazole, pH 8) for 1 h at 4° C. with gentle agitation, followed by 800×g centrifugation for 1 min and the supernatant was kept as ‘flow through’. After washing 4× (using the binding buffer but with 15 mM imidazole), the resins were incubated with 8 ml of elution buffer (same as binding buffer but with 200 mM imidazole) for 10 min at 4° C. with gentle agitation, followed by collection of the eluates using 5 ml-column (Evergreen Scientific). After measuring the protein concentration, the eluates were enriched with 10% glycerol and 1 mM DTT and stored at −80° C. The concentrations of recombinant proteins added to hepatocytes or macrophages was 0.5 μg/ml (rTF) or 1 μg/ml (rCPS1), similar to the ratio's used in the animal experiments.

For the lentiviral constructs, pLenti-lox-hCPS1-GFP was constructed using the Gibson assembly cloning method, and lentivirus was amplified and purified by the Vector core (University of Michigan Medical School).

### Measurement of CPS1 Activity

CPS1 enzymatic activity was measured using the hydroxyurea method (Pierson D L (1980) J Biochem Biophys Methods. 3, 31-37.; herein incorporated by reference in its entirety). The recombinant protein samples (15 μg) were incubated in 200 μl of reaction buffer [50 mM NH₄HCO₃, 5 mM ATP, 10 mM Mg(CH₃COO)₂, 5 mM N-acetyl-L-glutamate, 1 mM DTT and 50 mM triethanolamine (pH 8)] at 37° C. for 10 min, which generates carbamoyl phosphate via the enzymatic action of CPS1. Then, 100 mM of hydroxylamine was added to the reaction and incubated (95° C., 10 min) to convert carbamoyl phosphate to hydroxyurea. The hydroxyurea was quantified by adding to 0.8 ml of chromogenic reagent composed with antipyrine and diacetyl monoxime (Sigma), and heating (15 min, 95° C.), followed by measurement of colorimetric absorbance at 458 nm [using carbamoyl phosphate (Sigma) as a standard].

### TUNEL Assay

Cell death was detected using ApopTag Red In Situ Apoptosis detection Kit (EMD Millipore). Deparaffinized liver sections were incubated in a humidified chamber at 37° C. with TdT enzyme solution for 1 h, and applied to anti-digoxigenin conjugate solution (rhodamine) for 30 min (22° C.) in the dark. After washing, the slides were mounted and images were acquired.

### Phagocytosis Assay

Phagocytic activity was detected using a Phagocytosis assay kit (Cayman). Mice were administered 0.1 ml of rTF or rCPS1 via tail vein injection, and after 24 h they were injected intraperitonealy with APAP to trigger hepatic Mϕ activation. Hepatic Mϕ were isolated 6 h post-APAP injection, and plated on 4-well chamber slides. After 24 h, the culture media was changed using fresh media containing latex beads-rabbit IgG-FITC complex (1:200 of beads to media volume, latex bead size=100 nm), and the cells were cultured (37° C., 2 h). After washing, the cells were permeabilized and incubated with anti-vimentin/Alexa Fluor 680 for hepatic Mϕ staining. After washing, the slides were mounted and images were acquired.

### Statistics

Data are presented as mean±standard error of the mean (SEM) and graphed using GraphPad Prism 7. Data are representative of multiple independent experiments. The statistical significance was compared using an unpaired two-tailed Student's t-test for single comparisons. p<0.05 was considered to be statistically significant and was compared as *p<0.05, **p<0.01, ***p<0.001.

### Results

### CPS1 is Released as a Soluble Multimeric Protein.

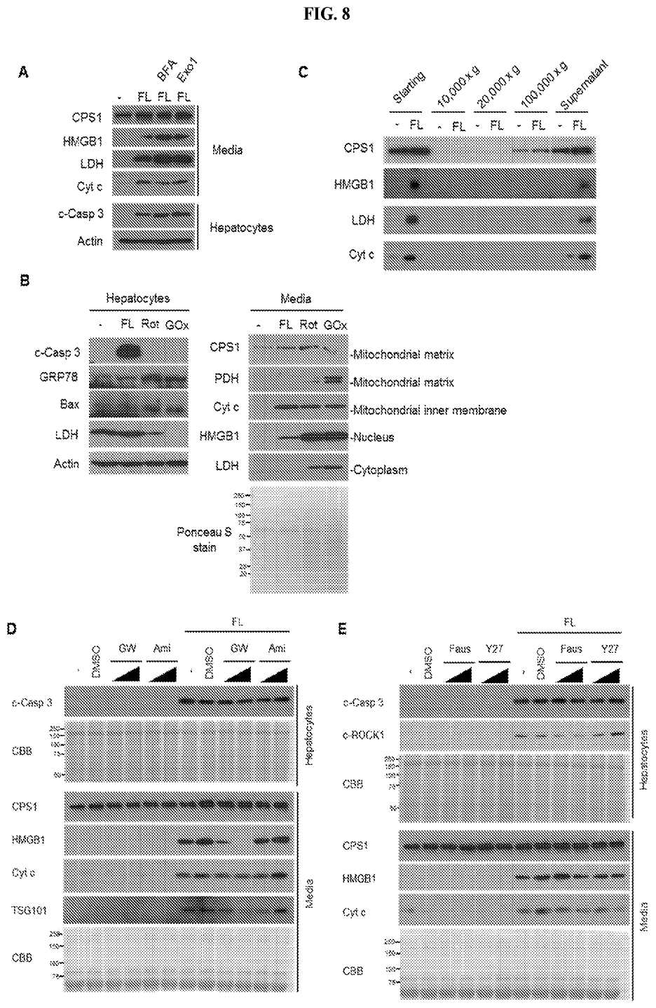

CPS1 is released into serum during liver injury (Weerasinghe S V, et al. (2014) Am J Physiol Gastrointest Liver Physiol 307:G355-364.; herein incorporated by reference in its entirety), and others found (using proteomic profiling) CPS1 in the extracellular vesicles (EVs) fraction secreted by rat primary hepatocytes (Conde-Vancells J et al. (2008) J Proteome Res 7:5157-5166.; herein incorporated by reference in its entirety). Nanoparticle tracking analysis (NTA), performed with culture media of mouse primary hepatocytes, showed that hepatocytes normally release EVs sized 102.8±1.9 nm in average, with a slight increase in size after incubation with FL (Fas ligand) and subsequent injury (FIG. 1A). The mechanism of CPS1 release during liver injury is unknown and CPS1 gene sequence does not contain a leader signal peptide for classic ER-Golgi-dependent secretory pathway. Indeed, inhibitors of classical exocytosis (brefeldin A, Exo1) did not block its release (FIG. 8A). CPS1 levels increased in hepatocyte culture media after incubation with FL along with DAMPs such as HMGB1, lactate dehydrogenase (LDH) and cytochrome c (FIG. 1B), but CPS1 was the major protein detected in the EV fraction collected by ultracentrifugation (100,000×g pellet) of the culture media (FIG. 1B). CPS1 release becomes enhanced not only by FL but also after incubation with rotenone or glucose oxidase, which increase intracellular oxidative stress and result in distinct release patterns for HMGB1, LDH or other mitochondrial proteins such as cytochrome c and pyruvate dehydrogenase (PDH) (FIG. 8B).

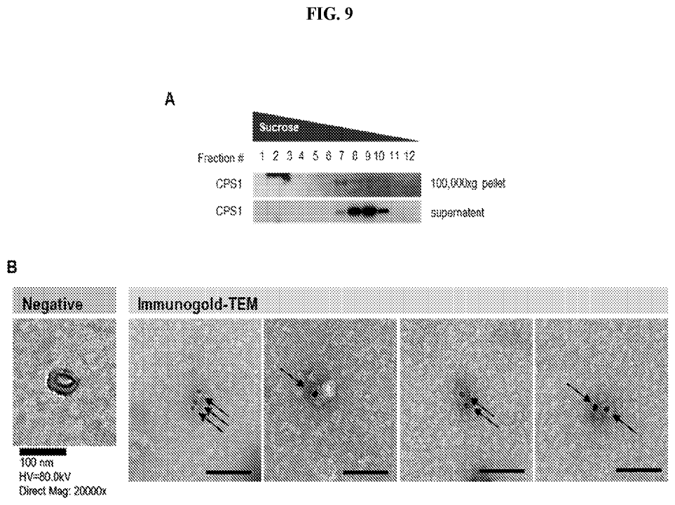

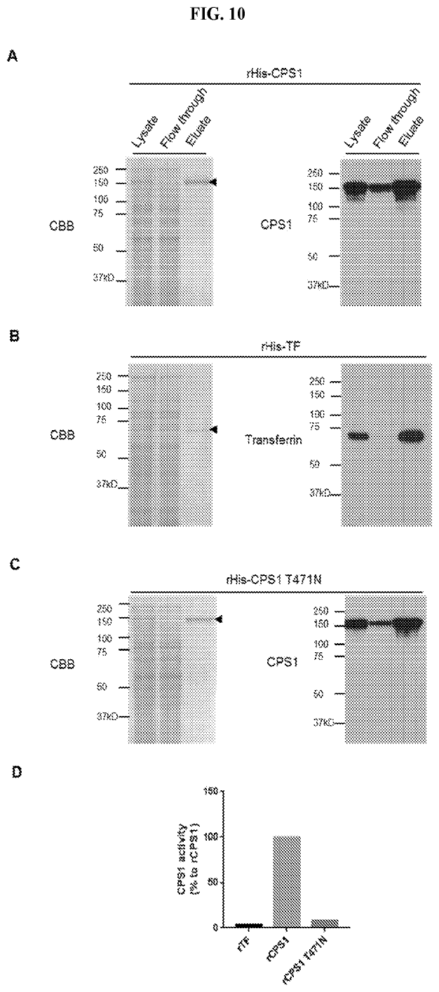

To examine the size of CPS1-containing EVs, culture media of hepatocytes or sera from mice given FL or APAP was pelleted at 10,000/20,000/100,000×g serially to enrich for apoptotic bodies, microvesicles or exosomes, respectively. Unlike HMGB1, LDH and cytochrome c, which were detected exclusively in the supernatant of FL-treated cells, CPS1 co-partitioned with the exosome-enriched fraction (100,000×g) and the supernatant ex vivo (FIG. 8C) and in vivo (FIG. 1C). Notably, none of these proteins was found in serum of healthy mice. Another mitochondrial matrix protein (PDH) and an outer membrane protein (Tom20) were observed in mouse sera independent on liver injury, but increased in the exosome fraction during liver injury (FIG. 1C). The exosome markers CD9 and TSG101 partitioned with the pellet as expected, but were also in the supernatant, suggesting leakage during fractionation or possibly being components of smaller vesicles that are not sedimented by 100,000×g centrifugation. However, incubation of hepatocytes with potential inhibitors for exosome secretion (GW4869 and amiloride) did not alter CPS1 release (FIG. 8D), nor did treatment with fausdil or Y-27632 [which inhibit Rho-associated, coiled-coil containing protein kinase (ROCK) signaling and modulate plasma membrane shedding] block CPS1 exocytosis (FIG. 8E). NTA analysis of culture media from hepatocytes transduced with CPS1-GFP showed that most of the CPS1-containing GFP-positive particles were smaller than 40 nm (mode: 35.3±0.6 nm, FIG. 1D), which is similar to the smallest size of the expected exosome size (Hirsova P et al. (2016) Gastroenterology 150: 956-967.; Raposo G and Stoorvogel W (2013) J Cell Biol 200:373-383.; herein incorporated by reference in their entireties). However, sucrose gradient separation showed that CPS1 was broadly detected in most of the fractions from the 100,000×g pellet isolated from hepatocyte culture media, whereas the exosome markers CD9, TSG101, Flotillin1, and Alix were exclusively in fractions #7-9 (FIG. 1E). In addition, sucrose gradient centrifugation of mice sera showed that CPS1 in the supernatant after 100,000×g spin sedimented in fraction #7-10 (FIG. 9A), thereby indicating that even soluble CPS1 forms multimers that co-sediment with EVs. Supporting this, electron microscopy of immunogold staining of CPS1 in the 100,000×g pellet of mouse serum showed immune reactivity with aggregate-like structures (FIG. 9B). Moreover, sucrose gradient separation of purified recombinant CPS1 (rCPS1) that we generated (FIG. 10A) showed a broad distribution consistent with formation of CPS1 multimers (FIG. 1F). Collectively, these data are consistent with CPS1 release from hepatocytes as a soluble protein that spontaneously form multimers, with sedimentation properties that overlap with EVs.

### CPS1 is Found in Normal Mouse and Human Bile.

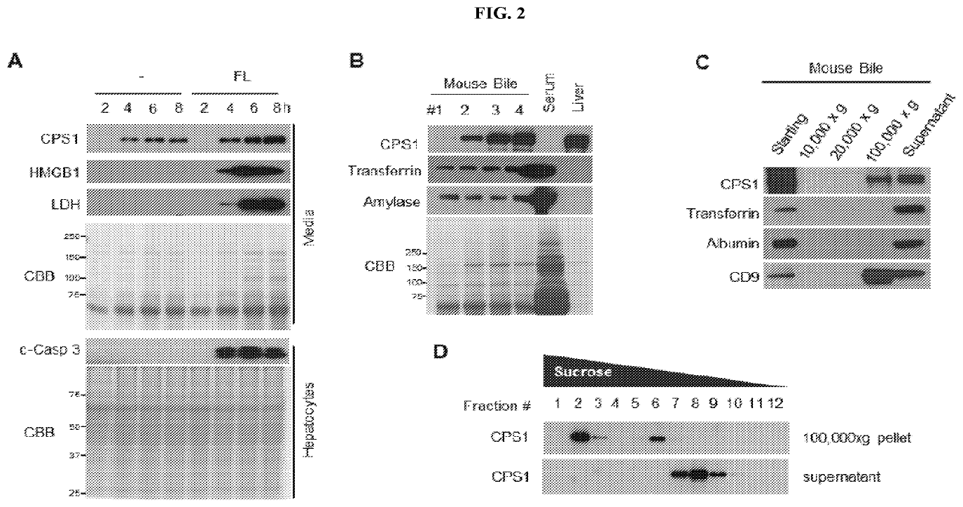

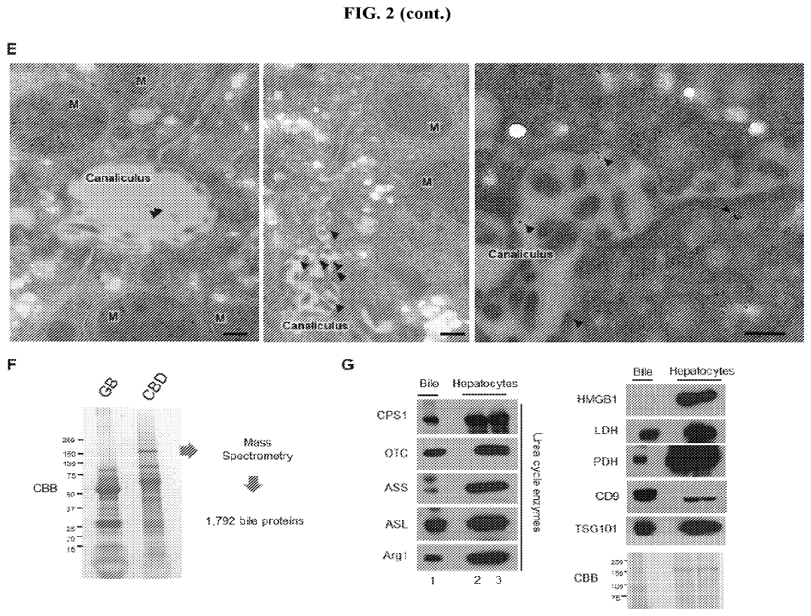

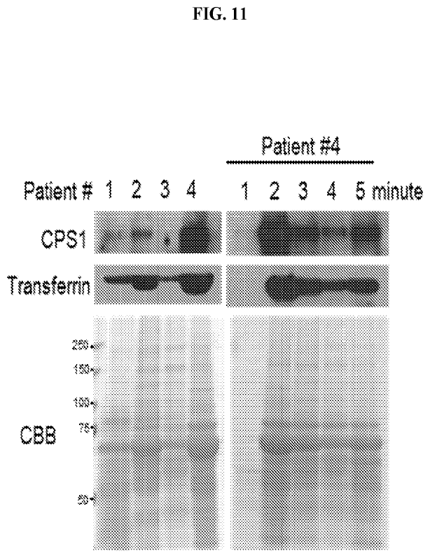

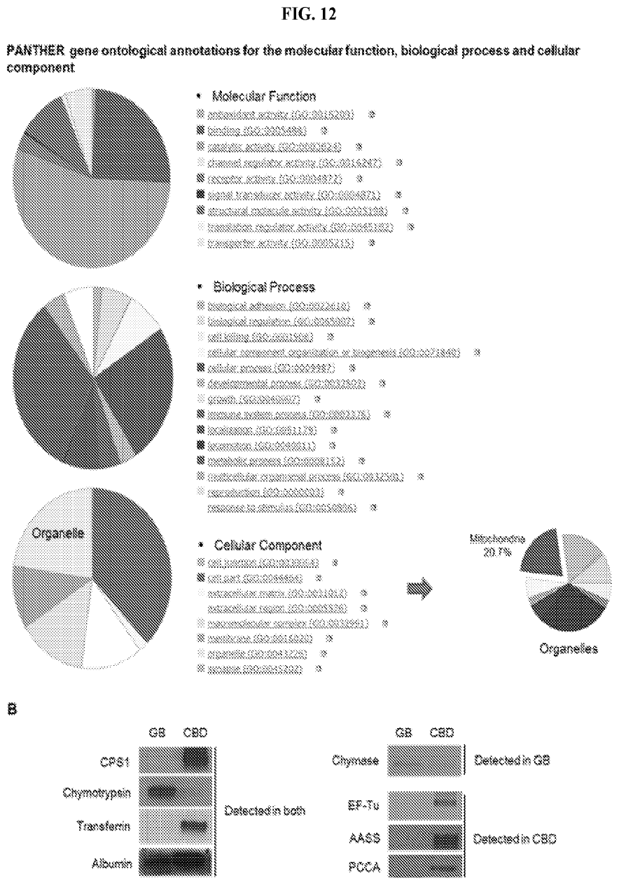

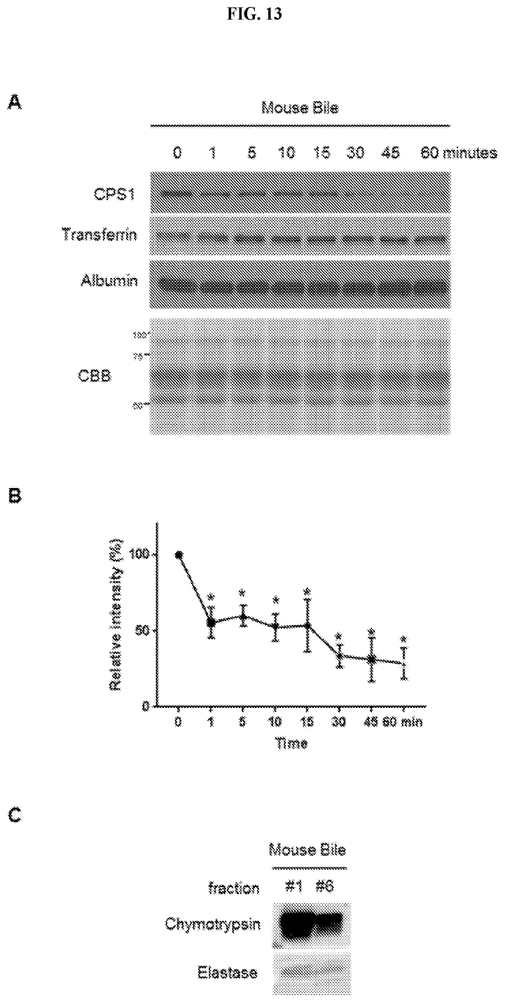

CPS1 is not observed in serum of healthy mice (FIG. 1C), but is readily detected in hepatocyte culture media in the absence of insults unlike HMGB1 and LDH (FIG. 1B, FIG. 2A). This discrepancy indicates that CPS1 may be normally secreted luminally into bile in the polarized hepatocytes in vivo. Bile we collected from the common bile duct of mice at 20-minute intervals. The collected bile showed high levels of CPS1, while no CPS1 was detected in serum (FIG. 2B). Transferrin and amylase were observed in mouse bile and serum as expected, consistent with the majority of bile proteins being derived from plasma (Mullock B M et al. (1985) Gut 26:500-509.; herein incorporated by reference in its entirety). Similar findings were noted in human bile samples collected from common bile duct, with some variability among individuals (FIG. 11). Consistent with the CPS1 in serum or culture media of hepatocytes, bile CPS1, unlike transferrin and albumin, was observed in the 100,000×g pellet fraction and the supernatant (FIG. 2C). CPS1 in the pelleted bile fraction was separated in the higher sucrose concentration fractions (FIG. 2D), but even CPS1 in the bile supernatant (FIG. 2D) sedimented in fractions similar to those seen in the supernatant fraction of mouse serum (FIG. 9A). Ultrastructural analysis using immunogold staining of normal mouse liver showed CPS1 within and near liver canaliculi by (FIG. 2E). Mass spectrometry of proteins from bile, obtained from gallbladder and common bile duct, identified 1,792 proteins (FIG. 2F, FIG. 12). Notably, many mitochondrial proteins and all five enzyme components of the urea cycle, in addition to LDH, PDH, and the two exosome markers, CD9 and TSG101, were found in bile (FIG. 2G). CPS1 had a shorter half-life in bile than transferrin and albumin, and gallbladder bile had detectable pancreatic enzymes (FIG. 13) which explains the near-absent CPS1 level in gallbladder bile (FIG. 12B). This data indicates that CPS1 is normally released to bile canaliculus via the hepatocyte apical membrane, but is re-routed to the sinusoids upon hepatotoxicity, thereby rendering it readily detectable in serum during liver injury.

### Uptake of Serum CPS1 by Macrophages

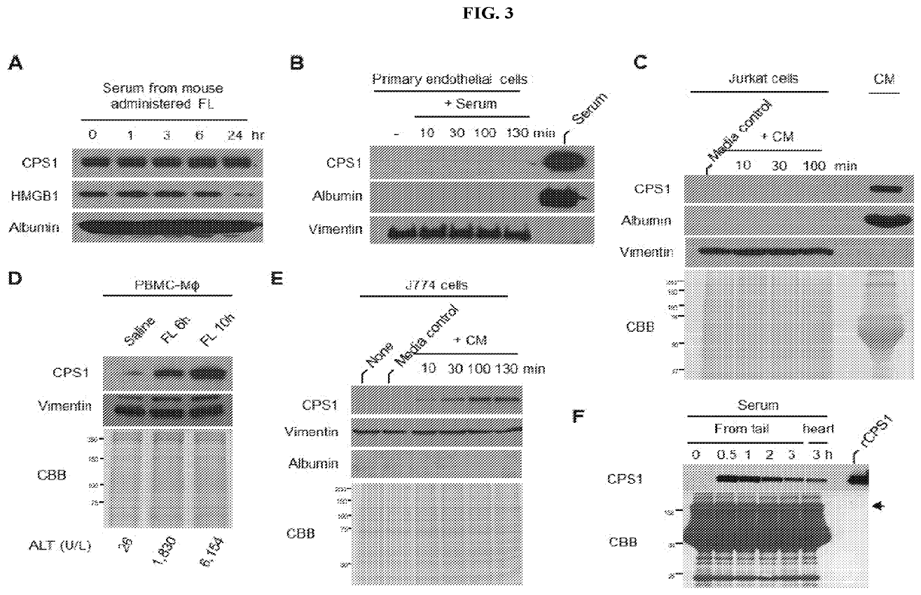

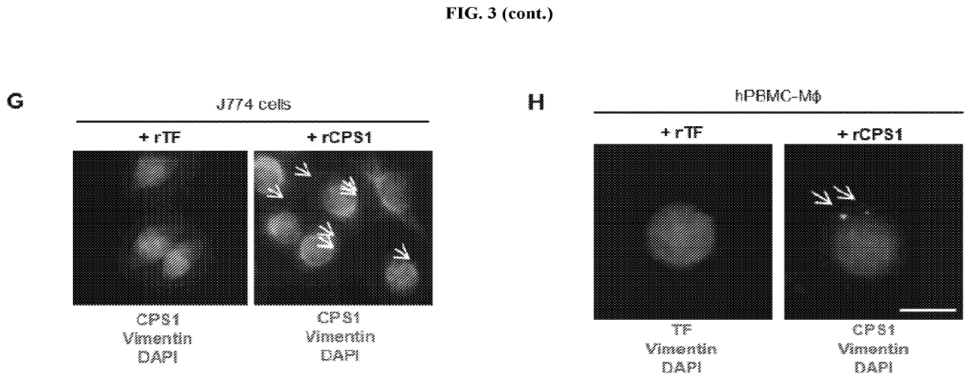

To investigate whether CPS1 is degraded by serum proteases, serum from the FL-treated mice was incubated (37° C.) and tested over time. Serum CPS1 was not degraded after 24 h, in contrast with HMGB1 (FIG. 3A), thereby indicating that serum proteases are not responsible for the rapid turnover of CPS1. It was then examined whether serum CPS1 is taken up by endothelial cells or leukocytes. Primary endothelial cells from mouse aorta or human Jurkat T cells did not take up CPS1 (FIG. 3B,C), while Mϕ from peripheral blood mononuclear cells (PBMC-Mϕ) of mice injected with FL accumulated CPS1 (FIG. 3D). Similarly, CPS1 was specifically taken up by the J774 Mϕ cell line incubated with hepatocyte culture media containing CPS1 (FIG. 3E). To further examine whether CPS1 is taken up by Mϕ, His-tagged full-length human rCPS1 and a human transferrin (rTF) variant (a mutant form unable to bind iron; as a control) were generated (FIG. 10A). Intravenous administration of rCPS1 into naïve mice showed fast in vivo turn-over rate (T½=58 minutes) (FIG. 3F), consistent with the observation of rapid endogenous CPS1 turnover in blood during acute liver injury (Weerasinghe S V, et al. (2014) Am J Physiol Gastrointest Liver Physiol 307:G355-364.) and rat CPS1 half-life of 67 minutes in blood (Ozaki M et al. (1994) Enzyme Protein. 48: 213-221.; herein incorporated by reference in its entirety). Immunofluorescence analysis showed rCPS1 uptake by J774 Mϕ and human PBMC-Mϕ (FIG. 3G,H) which supports the rapid clearance of CPS1 in vivo.

### CPS1 Induces M2 Polarization of Monocytes and Hepatic Macrophages, Independent of its Enzyme Activity

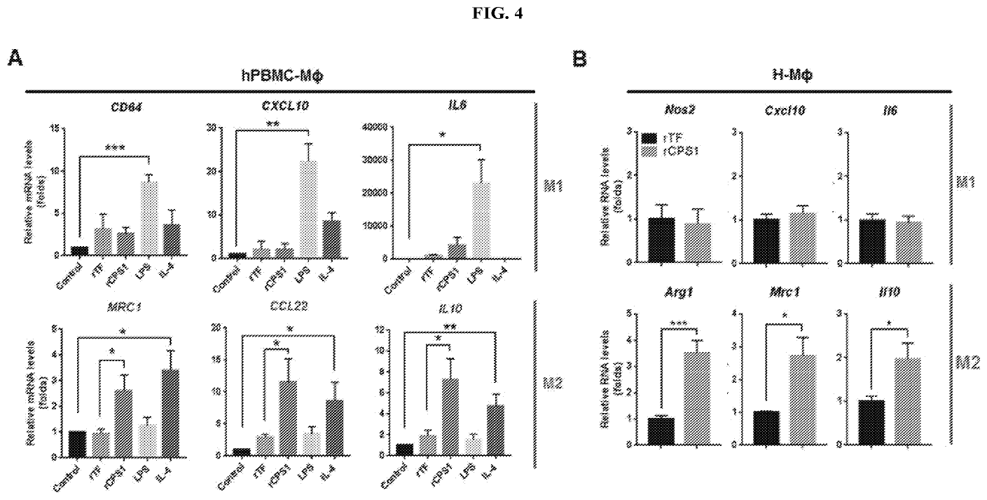

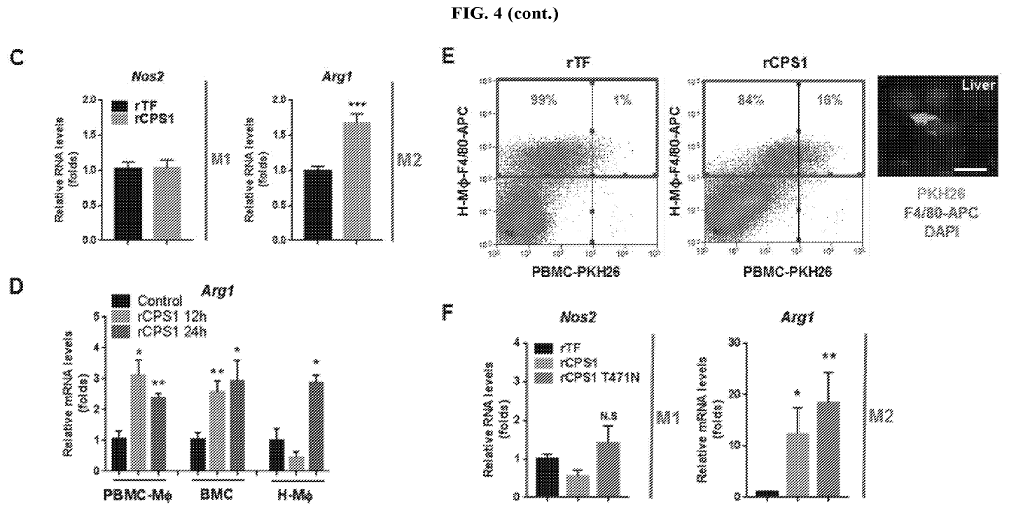

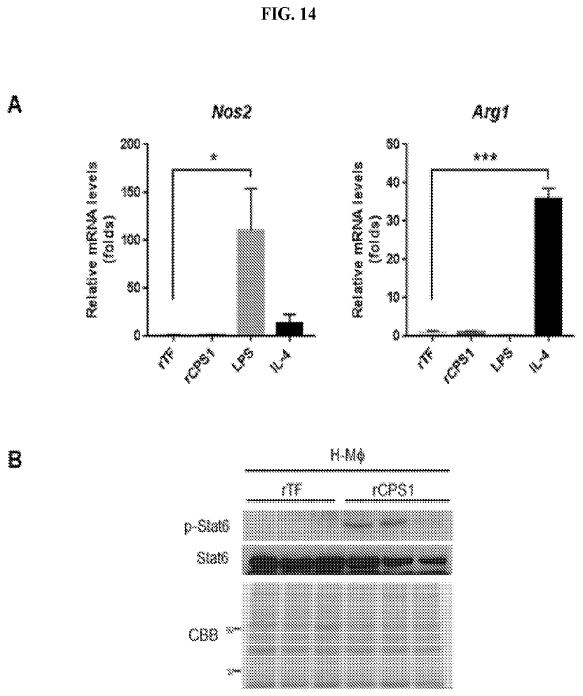

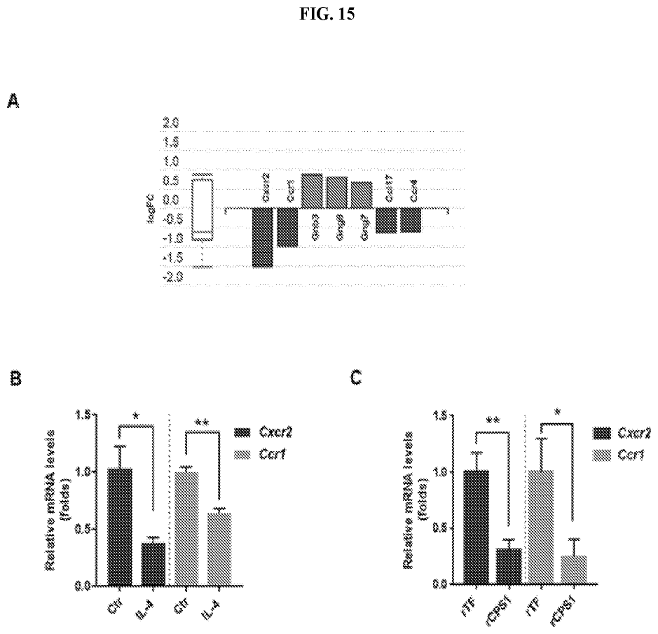

Hepatic Mϕs are comprised of liver-resident Kupffer cells, or bone marrow-derived monocytes recruited under liver disease conditions, and these cells actively participate in liver homeostasis (Krenkel O and Tacke F (2017) Nat Rev Immunol 17:306-321.; herein incorporated by reference in its entirety). Given CPS1 uptake by monocytes/Mϕ, experiments were conducted during development of embodiments herein to examine whether CPS1 activates Mϕ via the classical (M1) or alternative (M2) modes (Sica A, et al. (2014) Hepatology 59:2034-2042.; herein incorporated by reference in its entirety). While expression of M1-related (CD64/CXCL10/IL6) or M2-related (MRC1/CCL22/IL10) genes were elevated after incubation with LPS or IL-4, respectively, rCPS1 but not rTF significantly increased M2 gene expression (FIG. 4A). In contrast, Arg1 expression was not altered by rCPS1 treatment of naïve Kupffer cells ex vivo (FIG. 14A). However, rCPS1 administration significantly increased M2 gene expression (Arg1/Mrc1/Il10) of hepatic Mϕ (FIG. 4B) in association with Stat6 phosphorylation (FIG. 14B). Moreover, transwell co-culture of isolated naïve Kupffer cells with PBMCs from mice administered rCPS1 showed that factors released by the PBMCs induce M2 polarization of Kupffer cells without needing direct contact (FIG. 4C). Notably, Cxcr2 and Ccr1 expression was the most reduced among the chemokine signaling pathway genes in hepatic Mϕ from the rCPS1-APAP-administered mice as compare to the rTF-APAP-administered mice (FIG. 15A). In line with the inflammatory roles of CXCR2 and CCR1 (Kuboki S et al. (2008) Hepatology 48:1213-1223.; Van Sweringen H L et al. (2013) Hepatology 57:331-338.; Ju C and Tacke F (2016) Cell Mol Immunol. 13:316-27.; herein incorporated by reference in their entireties), their gene expression was decreased by IL-4 treatment and their down-regulation by rCPS1 was validated by independent qPCR of hepatic Mϕ from mice injected with rCPS1 or rTF (FIG. 15B,C). These results indicate that CPS1 in serum elicits an anti-inflammatory role via Mϕ during liver injury.

Given the heterogeneity of hepatic Mϕ, it was examined if CPS1 could promote recruitment of circulating monocytes to the liver. PBMC-Mϕ, bone marrow cells and hepatic Mϕ, isolated from the same mice 12 h or 24 h post-administration of recombinant proteins, showed that Arg1 expression in PBMC-Mϕ and bone marrow cells peaked much earlier (at 12 h), while hepatic Mϕ activation followed at 24 h (FIG. 4D). Homing to the liver was validated by isolating PBMC-Mϕ from mice injected with rTF or rCPS1, labeling with PKH26, then re-injecting into mice followed by isolation of the hepatic Mϕs to test for the presence of labeled cells (FIG. 16). Notably, 16% of the terminally isolated F4/80+ hepatic Mϕs harbored PKH26 dye (e.g., representing PBMC-Mϕ from mice preactivated with rCPS1 that homed to the liver), while only 1% of the cells pre-activated with rTF co-stained with PKH26 (FIG. 4E). Hence, CPS1 elicits PBMC-MϕM2-polarization in blood or bone marrow, with subsequent homing of these activated cells to the liver. The rCPS1 T471N mutant (FIG. 10C,D), which is enzymatically inactive (Pekkala S et al. (2010) Hum Mutat 31:801-808.; herein incorporated by reference in its entirety), did not alter the effect of CPS1 on M2 gene expression (FIG. 4F), thereby indicating that the cytokine-like role of CPS1 is independent of its enzymatic activity.

Prophylactic and Therapeutic Effects of rCPS1 in Experimental Liver Injury

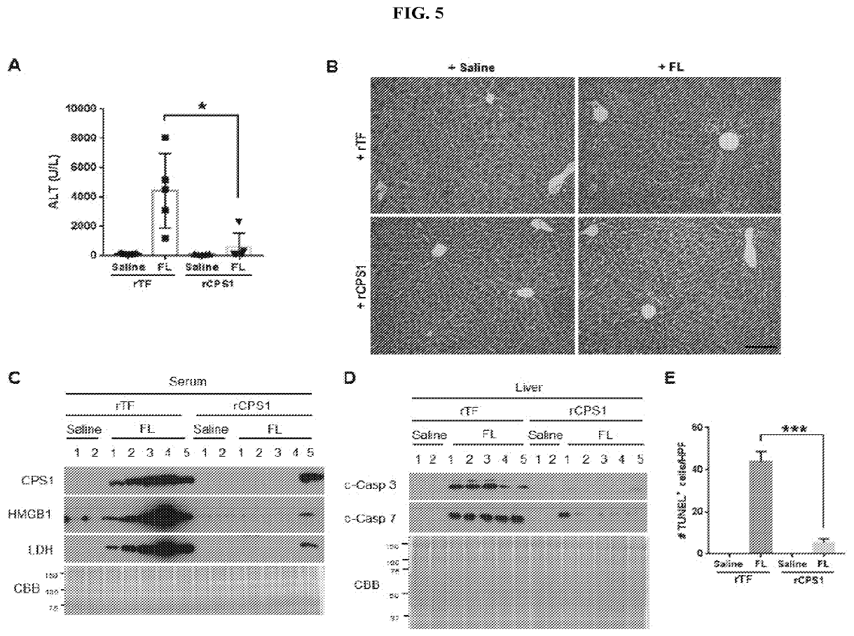

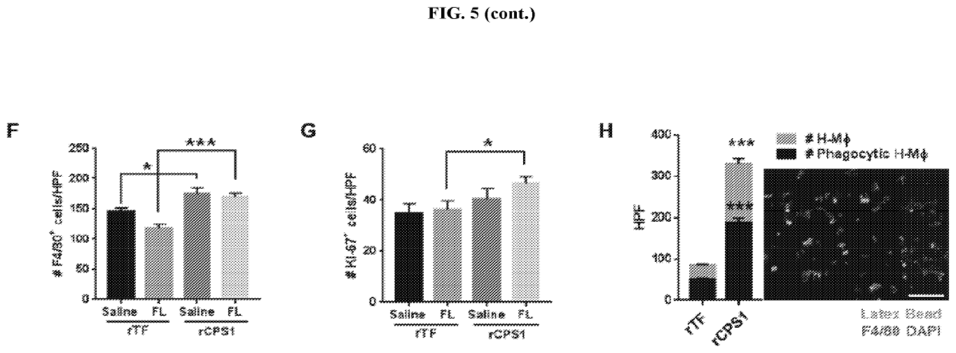

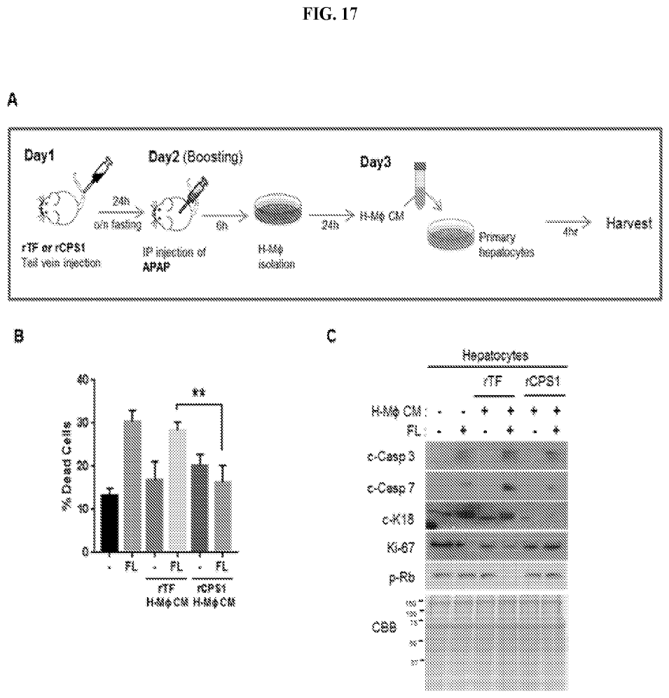

Contrary to the pro-inflammatory M1 Mϕs, the anti-inflammatory M2 Mϕs are involved in repair and proliferation (Sica A, et al. (2014) Hepatology 59:2034-2042.). Thus, experiments were conducted during development of embodiments herein to examine whether CPS1 has a protective role during liver injury. Mice were given rTF or rCPS1 then injected with saline or FL after 24 h. rTF-FL-administered mice had elevated alanine transaminase (ALT), cell death and liver hemorrhage as expected, whereas rCPS1-FL-administered mice had limited ALT elevation and significantly less histologic liver damage (FIG. 5A,B). Release of CPS1/HMGB1/LDH were greatly attenuated in rCPS1-FL mice, compared with rTF-FL mice, along with decreased apoptosis in the livers (determined by cleaved caspase-3 and -7 and TUNEL staining, FIG. 5C-E). The F4/80+Mϕ number increased significantly in livers of mice given rCPS1, coupled with an increased Ki-67+ cells (FIG. 5F,G). In addition, rCPS1 led to >3-fold increase in phagocytic activity of hepatic Mϕ (FIG. 5H), which may contribute to debris clearance. Hence, rCPS1 leads hepatic Mϕs to proliferate and undergo M2 polarization to an anti-inflammatory phenotype. The CPS1 protective effect is likely mediated by Mϕ cytokines, since hepatic Mϕ-conditioned media isolated from rCPS1-injected, but not rTF-injected, mice decreased hepatocyte cell death and elevated Ki-67 and phosphorylated-Rb upon FL treatment (FIG. 17).

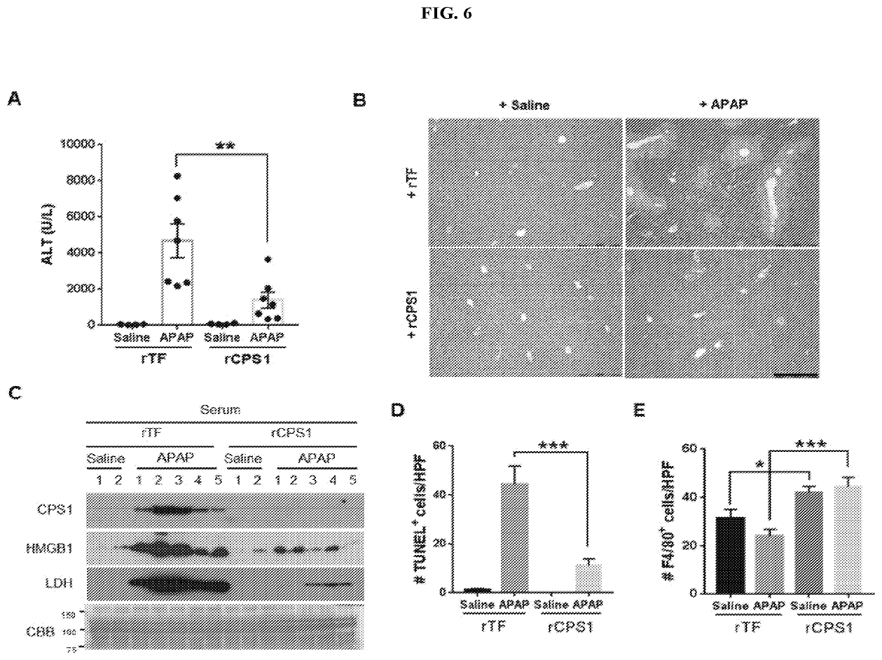

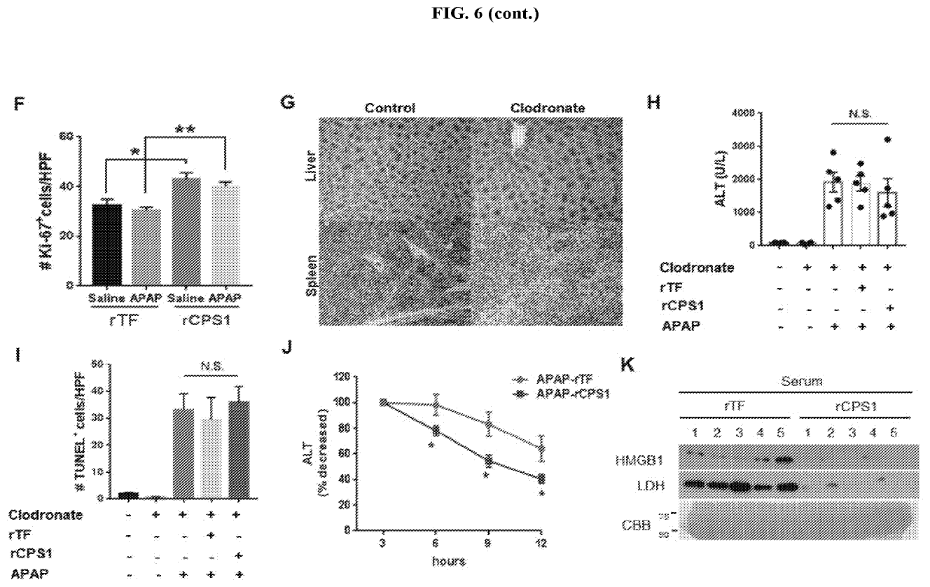

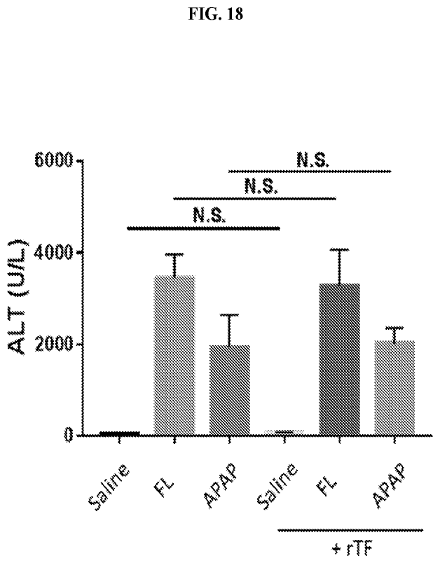

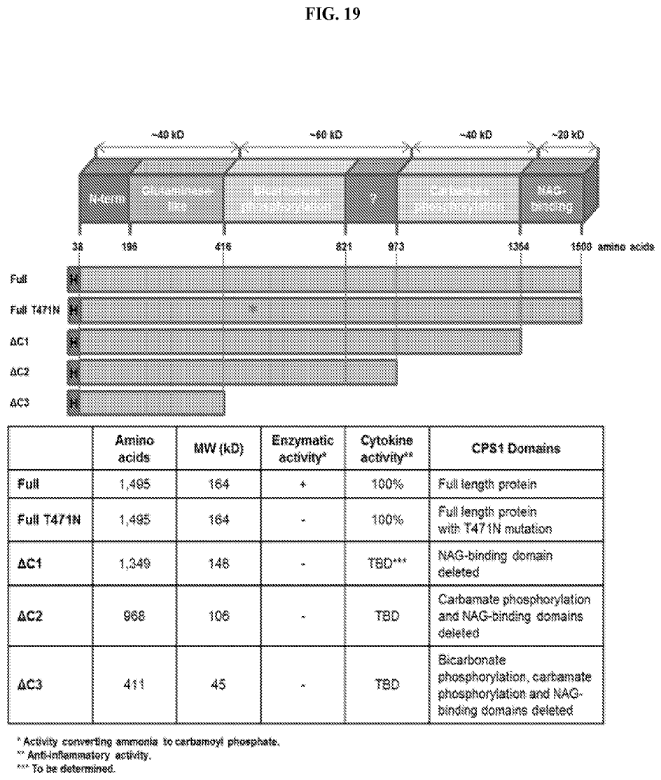

Experiments were conducted during development of embodiments herein to test the effect of CPS1 on APAP-induced liver injury, which closely mimics human drug-induced liver injury. Administration of rCPS1 24 h before exposure to APAP attenuated liver damage significantly as determined by serum ALT, liver histology, and serum levels of CPS1/HMGB1/LDH (FIG. 6A-C). TUNEL+ cells were decreased while F4/80+Mϕ and Ki-67+ cells increased upon APAP exposure in the rCPS1 versus the control rTF group (FIG. 6D-F). rTF alone does not impact the extent of FL- or APAP-induced liver injury (FIG. 18) thereby indicating that the protective effect imparted by rCPS1 is not related to a damaging effect that is mediated by rTF. Experiments were conducted during development of embodiments herein to test the importance of macrophages in the observed CPS1 protective effect. For this, macrophages were depleted using clodronate liposome administration (FIG. 6G), and this depletion blocked the protective effect of CPS1 as determined by serum ALT and TUNEL staining analysis (FIG. 6H,I). This provides indicates that CPS1 attenuation of liver injury occurs through Mϕs.

Administration of rCPS1 3 h post-APAP exposure, when serum ALT levels are highly elevated (average ALT>2,000), also led to more rapid recovery from liver injury as compared with rTF-injected mice (FIG. 6J). Consistent with this, serum HMGB1 and LDH were markedly lower in sera of APAP-rCPS1 mice compared to APAP-rTF mice (FIG. 6K). These overall findings indicate that CPS1 serves as an anti-inflammatory cytokine that provides therapeutic benefit in the setting of acute liver injury (FIG. 7).

### REFERENCES

The following references, some of which are also cited above, are herein incorporated by reference in their entireties.
Bernal W and Wendon J (2013) Acute Liver Failure. N Engl J Med 369:2525-2534.
Lee W M (2013) Drug-induced Acute Liver Failure. Clin Liver Dis 17, 575-86.
Antoine D J et al. (2012) Molecular forms of HMGB1 and keratin-18 as mechanistic biomarkers for mode of cell death and prognosis during clinical acetaminophen hepatotoxicity. J Hepatol 56:1070-1079.
Ilmakunnas M. et al. (2008) High mobility group box 1 protein as a marker of hepatocellular injury in human liver transplantation. Liver Transpl 14:1517-1525.
Kostova N, Zlateva S, Ugrinova I, and Pasheva E (2010). The expression of HMGB1 protein and its receptor RAGE in human malignant tumors. Mol Cell Biochem 337:251-258.
Yan W et al. (2012) High-mobility group box 1 activates caspase-1 and promotes hepatocellular carcinoma invasiveness and metastases. Hepatology 55:1863-1875.
Bianchi M E et al. (2017) High-mobility group box 1 protein orchestrates responses to tissue damage via inflammation, innate and adaptive immunity, and tissue repair. Immunol Rev 280:74-82.
Eleftheriadis T, Pissas G, Liakopoulos V, and Stefanidis I (2016) Cytochrome c as a Potentially Clinical Useful Marker of Mitochondrial and Cellular Damage. Front Immunol 7:279.
Miller T J et al. (2008) Cytochrome c: a non-invasive biomarker of drug-induced liver injury. J Appl Toxicol 28:815-828.
Chen G Y, and Nunez G (2010) Sterile inflammation: sensing and reacting to damage. Nat Rev Immunol 10:826-837.
Szabo G and Petrasek J (2015) Inflammasome activation and function in liver disease. Nat Rev Gastroenterol Hepatol 12:3 87-400.
Weerasinghe S V, Jang Y J, Fontana R J, and Omary M B (2014) Carbamoyl phosphate synthetase-1 is a rapid turnover biomarker in mouse and human acute liver injury. Am J Physiol Gastrointest Liver Physiol 307:G355-364.
Clarke S (1976) A major polypeptide component of rat liver mitochondria: carbamyl phosphate synthetase. J Biol Chem. 251:950-961.
Nicoletti M, Guerri C, and Grisolia S (1977) Turnover of carbamyl-phosphate synthase, of other mitochondrial enzymes and of rat tissues. Effect of diet and of thyroidectomy. Eur J Biochem. 75:583-592.
de Cima S et al. (2015) Structure of human carbamoyl phosphate synthetase: deciphering the on/off switch of human ureagenesis. Sci Rep 5:16950.
Pekkala S et al. (2010) Understanding carbamoyl-phosphate synthetase I (CPS1) deficiency by using expression studies and structure-based analysis. Hum Mutat 31:801-808.
Ozaki M et al. (1994) Enzyme-linked immunosorbent assay of carbamoyl phosphate synthetase 1: plasma enzyme in rat experimental hepatitis and its clearance. Enzyme Protein. 48: 213-221
Kim J et al. (2017) CPS1 maintains pyrimidine pools and DNA synthesis in KRAS/LKB1-mutant lung cancer cells. Nature 546:168-172.
Colombo M, Raposo G and Thery C (2014) Biogenesis, secretion, and intercellular interactions of exosomes and other extracellular vesicles. Annu Rev Cell Dev Biol 30:255-289.
Maas S L, Breakefield X O and Weaver A M (2017) Extracellular Vesicles: Unique Intercellular Delivery Vehicles. Trends Cell Biol 27:172-188.
Hirsova P et al. (2016) Lipid-Induced Signaling Causes Release of Inflammatory Extracellular Vesicles From Hepatocytes. Gastroenterology 150: 956-967.
Raposo G and Stoorvogel W (2013) Extracellular vesicles: exosomes, microvesicles, and friends. J Cell Biol 200:373-383.
Aicart-Ramos C, Valero R A and Rodriguez-Crespo I (2011) Protein palmitoylation and subcellular trafficking. Biochim Biophys Acta 1808:2981-2994.
Resh M D (1999) Fatty acylation of proteins: new insights into membrane targeting of myristoylated and palmitoylated proteins. Biochim Biophys Acta 1451:1-16.
Sugiura A, McLelland G L, Fon E A and McBride H M (2014) A new pathway for mitochondrial quality control: mitochondrial-derived vesicles. EMBO J 33:2142-2156.
Mullock B M et al. (1985) Sources of proteins in human bile. Gut 26:500-509.
Barbhuiya M A et al. (2011) Comprehensive proteomic analysis of human bile. Proteomics 11:4443-4453.
Guerrier L et al. (2007) Contribution of solid-phase hexapeptide ligand libraries to the repertoire of human bile proteins. J Chromatogr A 1176:192-205.
Zhang D et al. (2013) Comparative proteomic analysis of gallbladder bile proteins related to cholesterol gallstones. PLoS One 8:e54489.
Zhou H et al. (2005) Large-scale identification of human biliary proteins from a cholesterol stone patient using a proteomic approach. Rapid Commun Mass Spectrom 19:3569-3578.
Krenkel O and Tacke F (2017) Liver macrophages in tissue homeostasis and disease. Nat Rev Immunol 17:306-321.
Sica A, Invernizzi P and Mantovani A (2014) Macrophage plasticity and polarization in liver homeostasis and pathology. Hepatology 59:2034-2042.
Kuboki S et al. (2008) Hepatocyte signaling through CXC chemokine receptor-2 is detrimental to liver recovery after ischemia/reperfusion in mice. Hepatology 48:1213-1223.
Van Sweringen H L et al. (2013) Roles of hepatocyte and myeloid CXC chemokine receptor-2 in liver recovery and regeneration after ischemia/reperfusion in mice. Hepatology 57:331-338.
Ju C and Tacke F (2016) Hepatic macrophages in homeostasis and liver diseases: from pathogenesis to novel therapeutic strategies. Cell Mol Immunol. 13:316-27.
Pratten M K and Lloyd J B (1986) Pinocytosis and phagocytosis: the effect of size of a particulate substrate on its mode of capture by rat peritoneal macrophages cultured in vitro. Biochim Biophys Acta. 881:307-313.
Conde-Vancells J et al. (2008) Characterization and comprehensive proteome profiling of exosomes secreted by hepatocytes. J Proteome Res 7:5157-5166.
Bobrie A, Colombo M, Krumeich S, Raposo G and Thery C. (2012) Diverse subpopulations of vesicles secreted by different intracellular mechanisms are present in exosome preparations obtained by differential ultracentrifugation. J Extracell Vesicles 1:18397.
Gallart-Palau X et al. (2015) Extracellular vesicles are rapidly purified from human plasma by PRotein Organic Solvent PRecipitation (PROSPR). Sci Rep 5:14664.
Kowal J et al. (2016) Proteomic comparison defines novel markers to characterize heterogeneous populations of extracellular vesicle subtypes. Proc Natl Acad Sci USA 113:E968-977.
Yuana Y et al. (2013) Cryo-electron microscopy of extracellular vesicles in fresh plasma. J Extracell Vesicles 2: 21494.
Maeda A and Fadeel B (2014) Mitochondria released by cells undergoing TNF-alpha-induced necroptosis act as danger signals. Cell Death Dis 5:e1312.
Zhang Q et al. (2010) Circulating mitochondrial DAMPs cause inflammatory responses to injury. Nature 464:104-107.
Davis C H et al. (2014) Transcellular degradation of axonal mitochondria. Proc Natl Acad Sci USA 111:9633-9638.
Falchi A M et al. (2013) Astrocytes shed large membrane vesicles that contain mitochondria, lipid droplets and ATP. Histochem Cell Biol 139:221-231.
Hayakawa K et al. (2016) Transfer of mitochondria from astrocytes to neurons after stroke. Nature 535:551-555.
Islam M N et al. (2012) Mitochondrial transfer from bone-marrow-derived stromal cells to pulmonary alveoli protects against acute lung injury. Nat Med 18:759-765.
Matheoud D et al. (2016) Parkinson's Disease-Related Proteins PINK1 and Parkin Repress Mitochondrial Antigen Presentation. Cell 166:314-327.
McLelland G L, Lee S A, McBride H M and Fon E A (2016) Syntaxin-17 delivers PINK1/parkin-dependent mitochondrial vesicles to the endolysosomal system. J Cell Biol 214:275-291.
Neuspiel M et al. (2008) Cargo-selected transport from the mitochondria to peroxisomes is mediated by vesicular carriers. Curr Biol 18:102-108.
McLelland G L, Soubannier V, Chen C X, McBride H M, and Fon E A (2014) Parkin and PINK1 function in a vesicular trafficking pathway regulating mitochondrial quality control. EMBO J 33:282-295 .
Trinh J and Farrer M (2013) Advances in the genetics of Parkinson disease. Nat Rev Neurol 9:445-454.
Farina A, Dumonceau J M and Lescuyer P (2009) Proteomic analysis of human bile and potential applications for cancer diagnosis. Expert Rev Proteomics 6:285-301.
Farina A et al. (2009) Proteomic analysis of human bile from malignant biliary stenosis induced by pancreatic cancer. J Proteome Res 8:159-169.
Kristiansen T Z et al. (2004) A proteomic analysis of human bile. Mol Cell Proteomics 3:715-728.
Reichard H and Reichard P (1958) Determination of ornithine carbamyl transferase in serum. J Lab Clin Med. 52:709-717.
Reichard H (1961) Ornithine carbamyl transferase activity in human serum in disease of the liver and the biliary system. J Lab Clin Med. 57:78-87.
Lorentz K, Niemann E, Jaspers G, Oltmanns D (1969) Enzymes in human bile. II. Enzyme content of liver- and gallbladder bile. Enzymol Biol Clin. 10:528-533.
Reichard H (1959) Ornithine carbamyl transferase in dog serum on intravenous injection of enzyme, choledochus ligation, and carbon tetrachloride poisoning. J Lab Clin Med. 53(3):417-25
Kalaitzakis E et al. (2006) Evaluation of ornithine carbamoyl transferase and other serum and liver-derived analytes in diagnosis of fatty liver and postsurgical outcome of left-displaced abomasum in dairy cows. J Am Vet Med Assoc. 229:1463-71.
Margulis L and Chapman M J (1998) Endosymbioses: cyclical and permanent in evolution. Trends Microbiol 6:342-345
Alderete J S, Gaines E L and Hudson N L (1978) Contents and implications of ammonia human and canine bile. Gastroenterology 75:173-176.
Gowda G A et al. (2006) One-step analysis of major bile components in human bile using 1H NMR spectroscopy. Lipids 41:577-589.
Masyuk A I et al. (2010) Biliary exosomes influence cholangiocyte regulatory mechanisms and proliferation through interaction with primary cilia. Am J Physiol Gastrointest Liver Physiol 299:G990-999.
Zhou Z, Xu M J and Gao B (2016) Hepatocytes: a key cell type for innate immunity. Cell Mol Immunol 13:301-315.
Antoniades C G et al. (2012) Source and characterization of hepatic macrophages in acetaminophen-induced acute liver failure in humans. Hepatology 56:735-746.
Holt M P, Cheng L and Ju C (2008) Identification and characterization of infiltrating macrophages in acetaminophen-induced liver injury. J Leukoc Biol 84:1410-1421.
Zigmond E et al. (2014) Infiltrating monocyte-derived macrophages and resident kupffer cells display different ontogeny and functions in acute liver injury. J Immunol 193:344-353.
Ju C et al. (2002) Protective role of Kupffer cells in acetaminophen-induced hepatic injury in mice. Chem Res Toxicol. 15:1504-1513.
You Q et al. (2013) Role of hepatic resident and infiltrating macrophages in liver repair after acute injury. Biochem Pharmacol. 86:836-843.
Kobayashi M, Inoue K, Warabi E, Minami T and Kodama T (2005) A simple method of isolating mouse aortic endothelial cells. J Atheroscler Thromb. 12, 138-142.
Amend S R, Valkenburg K C and Pienta K J (2016) Murine Hind Limb Long Bone Dissection and Bone Marrow Isolation. J Vis Exp. 110.
Thery C, Amigorena S, Raposo G and Clayton A (2006) Isolation and characterization of exosomes from cell culture supernatants and biological fluids. Curr Protoc Cell Biol. Chapter 3, Unit 3 22.
Diez-Fernandez C, Hu L, Cervera J, Haberle J, and Rubio V (2014) Understanding carbamoyl phosphate synthetase (CPS1) deficiency by using the recombinantly purified human enzyme: effects of CPS1 mutations that concentrate in a central domain of unknown function. Mol Genet Metab 112:123-132.
Pierson D L (1980) A rapid colorimetric assay for carbamyl phosphate synthetase I. J Biochem Biophys Methods. 3, 31-37.

SEQUENCES
Full-length CPS1 (Homo sapiens)
SEQ ID NO: 1
MTRILTAFKVVRTLKTGFGFTNVTAHQKWKFSRPGIRLLSVKAQTAHIVLEDGTKMKG
YSFGHPSSVAGEVVFNTGLGGYPEAITDPAYKGQILTMANPIIGNGGAPDTTALDELGLS
KYLESNGIKVSGLLVLDYSKDYNHWLATKSLGQWLQEEKVPAIYGVDTRMLTKIIRDK
GTMLGKIEFEGQPVDFVDPNKQNLIAEVSTKDVKVYGKGNPTKVVAVDCGIKNNVIRLL
VKRGAEVHLVPWNHDFTKMEYDGILIAGGPGNPALAEPLIQNVRKILESDRKEPLFGIST
GNLITGLAAGAKTYKMSMANRGQNQPVLNITNKQAFITAQNHGYALDNTLPAGWKPLF
VNVNDQTNEGIMHESKPFFAVQFHPEVTPGPIDTEYLFDSFFSLIKKGKATTITSVLPKPA
LVASRVEVSKVLILGSGGLSIGQAGEFDYSGSQAVKAMKEENVKTVLMNPNIASVQTNE
VGLKQADVYFLPITPQFVTEVIKAEQPDGLILGMGGQTALNCGVELFKRGVLKEYGVKV
LGTSVESIMATEDRQLFSDKLNEINEKIAPSFAVESIEDALKAADTIGYPVMIRSAYALGG
LGSGICPNRETLMDLSTKAFAMTNQILVEKSVTGWKEIEYEVVRDADDNCVTVCNMEN
VDAMGVHTGDSVVVAPAQTLSNAEFQMLRRTSINVVRHLGIVGECNIQFALHPTSMEY
CIIEVNARLSRSSALASKATGYPLAFIAAKIALGIPLPEIKNVVSGKTSACFEPSLDYMVTK
IPRWDLDRFHGTSSRIGSSMKSVGEVMAIGRTFEESFQKALRMCHPSIEGFTPRLPMNKE
WPSNLDLRKELSEPSSTRIYAIAKAIDDNMSLDEIEKLTYIDKWFLYKMRDILNMEKTLK
GLNSESMTEETLKRAKEIGFSDKQISKCLGLTEAQTRELRLKKNIHPWVKQIDTLAAEYP
SVTNYLYVTYNGQEHDVNFDDHGMMVLGCGPYHIGSSVEFDWCAVSSIRTLRQLGKK
TVVVNCNPETVSTDFDECDKLYFEELSLERILDIYHQEACGGCIISVGGQIPNNLAVPLYK
NGVKIMGTSPLQIDRAEDRSIFSAVLDELKVAQAPWKAVNTLNEALEFAKSVDYPCLLR
PSYVLSGSAMNVVFSEDEMKKFLEEATRVSQEHPVVLTKFVEGAREVEMDAVGKDGR
VISHAISEHVEDAGVHSGDATLMLPTQTISQGAIEKVKDATRKIAKAFAISGPFNVQFLV
KGNDVLVIECNLRASRSFPFVSKTLGVDFIDVATKVMIGENVDEKHLPTLDHPIIPADYV
AIKAPMFSWPRLRDADPILRCEMASTGEVACFGEGIHTAFLKAMLSTGFKIPQKGILIGIQ
QSFRPRFLGVAEQLHNEGFKLFATEATSDWLNANNVPATPVAWPSQEGQNPSLSSIRKLI
RDGSIDLVINLPNNNTKFVHDNYVIRRTAVDSGIPLLTNFQVTKLFAEAVQKSRKVDSKS
LFHYRQYSAGKAA
CPS1 (Homo sapiens) H domain
SEQ ID NO: 2
MTRILTAFKVVRTLKTGFGFTNVTAHQKWKFSRPGIRL
CPS1 (Homo sapiens) N-terminal domain
SEQ ID NO: 3
LSVKAQTAHIVLEDGTKMKGYSFGHPSSVAGEVVFNTGLGGYPEAITDPAYKGQILTMA
NPIIGNGGAPDTTALDELGLSKYLESNGIKVSGLLVLDYSKDYNHWLATKSLGQWLQEE
KVPAIYGVDTRMLTKIIRDKGTMLGKIEFEGQPVDFVDP
CPS1 (Homo sapiens) Glutaminase-like domain
SEQ ID NO: 4
NKQNLIAEVSTKDVKVYGKGNPTKVVAVDCGIKNNVIRLLVKRGAEVHLVPWNHDFT
KMEYDGILIAGGPGNPALAEPLIQNVRKILESDRKEPLFGISTGNLITGLAAGAKTYKMS
MANRGQNQPVLNITNKQAFITAQNHGYALDNTLPAGWKPLFVNVNDQTNEGIMHESKP
FFAVQFHPEVTPGPIDTEYLFDSFFSLIKKGKATTITSVLPKPAL
CPS1 (Homo sapiens) Bicarbonate phosphorylation domain
SEQ ID NO: 5
VASRVEVSKVLILGSGGLSIGQAGEFDYSGSQAVKAMKEENVKTVLMNPNIASVQTNE
VGLKQADTVYFLPITPQFVTEVIKAEQPDGLILGMGGQTALNCGVELFKRGVLKEYGVK
VLGTSVESIMATEDRQLFSDKLNEINEKIAPSFAVESIEDALKAADTIGYPVMIRSAYALG
GLGSGICPNRETLMDLSTKAFAMTNQILVEKSVTGWKEIEYEVVRDADDNCVTVCNME
NVDAMGVHTGDSVVVAPAQTLSNAEFQMLRRTSINVVRHLGIVGECNIQFALHPTSME
YCIIEVNARLSRSSALASKATGYPLAFIAAKIALGIPLPEIKNVVSGKTSACFEPSLDYMVT
KIPRWDLDRFHGTSSRIGSSMKSVGEVMAIGRTFEESFQKALRMCHPSI
CPS1 (Homo sapiens) Central domain
SEQ ID NO: 6
EGFTPRLPMNKEWPSNLDLRKELSEPSSTRIYAIAKAIDDNMSLDEIEKLTYIDKWFLYK
MRDILNMEKTLKGLNSESMTEETLKRAKEIGFSDKQISKCLGLTEAQTRELRLKKNIHPW
VKQIDTLAAEYPSVTNYLYVTYNGQEHDVNFD
CPS1 (Homo sapiens) Carbamate phosphorylation domain
SEQ ID NO: 7
DHGMMVLGCGPYHIGSSVEFDWCAVSSIRTLRQLGKKTVVVNCNPETVSTDFDECDKL
YFEELSLERILDIYHQEACGGCIISVGGQIPNNLAVPLYKNGVKIMGTSPLQIDRAEDRSIF
SAVLDELKVAQAPWKAVNTLNEALEFAKSVDYPCLLRPSYVLSGSAMNVVFSEDEMK
KFLEEATRVSQEHPVVLTKFVEGAREVEMDAVGKDGRVISHAISEHVEDAGVHSGDAT
LMLPTQTISQGAIEKVKDATRKIAKAFAISGPFNVQFLVKGNDVLVIECNLRASRSFPFVS
KTLGVDFIDVATKVMIGENVDEKHLPTLDHPIIPADYVAIKAPMFSWPRLRDADPILRCE
MASTGEVACFGEGIHTAFLKAMLSTGFKIPQKGIL
CPS1 (Homo sapiens) NAG-binding domain
SEQ ID NO: 8
IGIQQSFRPRFLGVAEQLHNEGFKLFATEATSDWLNANNVPATPVAWPSQEGQNPSLSSI
RKLIRDGSIDLVINLPNNNTKFVHDNYVIRRTAVDSGIPLLTNFQVTKLFAEAVQKSRKV
DSKSLFHYRQYSAGKAA

1. A composition comprising a CPS1 polypeptide having at least 70% sequence identity to all or a portion of SEQ ID NO: 1, wherein the composition is not a product of nature, and wherein the CPS1 polypeptide exhibits a cytokine-like activity of wild-type of CPS1.

2. The composition of claim 1, wherein the CPS1 polypeptide comprises at least 70% sequence identity to one or a combination of SEQ ID NOs: 2-8.

3. The composition of claim 1, wherein the CPS1 polypeptide lacks a portion comprising 25% or greater sequence identity to one or more of SEQ ID NOs: 2-8.

4. A composition comprising a CPS1 peptide having at least 70% sequence identity a portion of SEQ ID NO: 1 that is 8-30 amino acid residues in length, wherein the composition is not a product of nature, and wherein the CPS1 peptide exhibits a cytokine-like activity of wild-type of CPS1.

5. The composition of one of claims 1-4, wherein the CPS1 peptide or polypeptide is fused to a second peptide or polypeptide.

6. The composition of claim 5, wherein the second peptide or polypeptide sequence is a carrier moiety, therapeutic moiety, or detectable moiety.

7. The composition of one of claims 1-6, wherein:
(i) one or more of the amino acid residues in the CPS1 peptide or polypeptide are D-enantiomers,
(ii) the CPS1 peptide or polypeptide comprises one or more unnatural amino acids,
(iii) the CPS1 peptide or polypeptide comprises one or more amino acid analogs, and/or
(iv) the CPS1 peptide or polypeptide comprises one or more peptoid amino acids.

8. The composition of one or claims 1-7, wherein the CPS1 peptide or polypeptide or an amino acid therein comprises a modification selected from the group consisting of phosphorylation, glycosylation, ubiquitination, S-nitrosylation, methylation, N-acetylation, C-terminal amidation, cyclization, substitution of natural L-amino acids with non-natural D-amino acids, lipidation, lipoylation, deimination, eliminylation, disulfide bridging, isoaspartate formation, racemization, glycation; carbamylation, carbonylation, isopeptide bond formation, sulfation, succinylation, S-sulfonylation, S-sulfinylation, S-sulfenylation, S-glutathionylation, pyroglutamate formation, propionylation, adenylylation, nucleotide addition, iodination, hydroxylation, malonylation, butyrylation, amidation, alkylation, acylation, biotinylation, carbamylation, oxidation, pegylation, and any other applicable peptide modification.

9. The composition of one of claim 1-7, wherein the CPS1 peptide or polypeptide exhibits enhanced stability, solubility, cytokine-like activity, cell permeability, and/or bioavailability relative to SEQ ID NO: 1.

10. The composition of one of claim 1-8, wherein the CPS1 peptide or polypeptide lacks CPS1 enzymatic activity.

11. A pharmaceutical composition comprising a composition of one of claims 1-10 and a pharmaceutically-acceptable carrier.

12. The pharmaceutical composition of claim 11, further comprising one or more additional therapeutic agents.

13. A method of treating or preventing acute internal tissue injury comprising administering to a subject a composition of one of claims 1-12.

14. The method of claim 13, wherein the subject suffers from a a disease that results in tissue injury.

15. The method of one of claims 13-14, wherein administering the composition increases macrophage numbers, phagocytic activity, and/or anti-inflammatory activity.

16. The method of one of claims 13-15, wherein administering the composition protects against tissue damage induced by a disease or condition.


SEQUENCE LISTING (8 sequences)

SEQ ID NO: 1 - protein, 1499 aa, Homo sapiens
MTRILTAFKVVRTLKTGFGFTNVTAHQKWKFSRPGIRLLSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 2 - protein, 38 aa, Homo sapiens
MTRILTAFKVVRTLKTGFGFTNVTAHQKWKFSRPGIRL

SEQ ID NO: 3 - protein, 157 aa, Homo sapiens
LSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDP

SEQ ID NO: 4 - protein, 220 aa, Homo sapiens
NKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPAL

SEQ ID NO: 5 - protein, 405 aa, Homo sapiens
VASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSI

SEQ ID NO: 6 - protein, 152 aa, Homo sapiens
EGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFD

SEQ ID NO: 7 - protein, 391 aa, Homo sapiens
DHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGIL

SEQ ID NO: 8 - protein, 137 aa, Homo sapiens
IGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA